(19) United States
(12) Patent Application Publication
(10) Pub. No.: US 2024/0327902 A1
(43) Pub. Date: Oct. 3, 2024

(54) METHODS FOR DETECTING AND ANALYSIS OF PROTEINS

(71) Applicant: Shifeng LI, Fremont, CA (US)

(72) Inventor: Shifeng LI, Fremont, CA (US)

(21) Appl. No.: 18/616,941

(22) Filed: Mar. 26, 2024

Related U.S. Application Data
(60) Provisional application No. 63/455,976, filed on Mar. 31, 2023.

(51) Int. Cl. C12Q 1/6834; C12Q 1/686; C12Q 1/6874; C12Q 1/6876

(52) U.S. Cl. CPC C12Q 1/6834; C12Q 1/686; C12Q 1/6874; C12Q 1/6876

(57) ABSTRACT

A method for detection and analysis of proteins includes the steps of: (a) providing a 1st capture molecule on 1st solid surfaces through a cleavable linker; (b) capturing a target protein with the 1st capture molecule on the 1st solid surfaces; (c) generating an attaching group on the captured protein without disrupting the captured protein complex; (d) cleaving the captured protein complex and immobilized on the 2nd solid surfaces through the attaching group on the captured protein; (e) releasing the 1 st capture molecule; (f) contacting the immobilized protein with 2nd capture molecule with a coding DNA tag; (g) transferring the DNA barcode in the coding DNA tag to a primer on the 2nd solid surfaces; (h) optionally amplifying the extended coding DNA strand into a cluster in situ on the 2nd solid surfaces; and (i) decoding DNA strands on the 2nd solid surfaces.

### CROSS-REFERENCE TO RELATED APPLICATIONS

The present application claims priority of U.S. Provisional Patent Application No. 63/455,976, filed Mar. 31, 2023, entitled “METHODS FOR DETECTION AND ANALYSIS OF PROTEINS” under 35 U.S.C. § 119(e), the disclosures of which are incorporated by reference in its entirety for all purposes.

### BACKGROUND OF THE INVENTION

### Field of the Invention

The present disclosure relates to methods for detection and analysis of proteins. In some embodiments, the methods can detect or analyze a pool of proteins from multiple samples.

### BRIEF DISCUSSION OF THE RELATED ART

Proteins are large biomolecules and macromolecules that comprise one or more long chains of amino acid residues. Like other biological macromolecules such as polysaccharides and nucleic acids, proteins are essential parts of organisms and participate in virtually every process within cells. Many proteins that catalyze biochemical reactions are vital to metabolism. Proteins also have structural or mechanical functions, such as actin and myosin in muscle and proteins in the cytoskeleton, which form a system of scaffolding that maintain cell shape. Other proteins are important in cell signaling, immune responses, cell adhesion, and the cell cycle. In animals, proteins are needed in the diet to provide the essential amino acids that cannot be synthesized. Digestion breaks the proteins down for metabolic use. Protein differs from one another primarily in their sequence of amino acids, which is dictated by the nucleotide sequence of their genes, and which usually results in protein folding into a specific 3D structure that determines its activity. Shortly after or even synthesis, the residues in a protein are often chemically modified by post translation modification (PTM), which alters physical and chemical properties, folding, stability, activity, and ultimately, the function of the proteins.

Proteins play an integral role in cell biology and physiology, performing and facilitating many different biological functions. The repertoire of different protein molecules is extensive, thus proteins contain a vast amount of information that is not largely unexplored. Yet the protein information is directly needed for a better understanding of proteome dynamics in health and disease and to help enable precision medicine. As such, there is a great need to develop high throughput tools to collect the vast amount of proteomic information.

Highly parallel protein characterization and recognition of proteins is challenging for several reasons. The use of affinity-based assays is often difficult due to several key challenges. One significant challenge is multiplexing the readout of a collection of affinity agents to a collection of cognate proteins; another challenging is minimizing cross reactivity between the affinity agents and off-target proteins; a third challenge is developing an efficient high throughput read out platform. Additionally, it is desirable to characterize various posttranslational modifications (PTMs) on the proteins at a single molecule level. Currently, this is a formidable task to accomplish in a high throughput way.

Molecular recognition and characterization of a protein or peptide is typically performed using an immunoassay. There are many different immunoassay formats including ELISA, multiplex ELISA (e.g., Quanterix, Singulex), reverse phase protein arrays, and many others. These different immunoassay platforms all face similar challenges including the development of high affinity and highly specific antibodies, limited ability to multiplex at both the sample and analyte level, limited sensitivity and dynamic range, and cross reactivity and background signals.

Recently, a few high throughput protein immunoassays achieved high specificity, high multiplex, high detection sensitivity and >10 logs dynamic range. Olink utilizes the combination of Proximity Extension Assay (PEA) and next generation sequencing to achieve high specific and multiplexed detection of up to 384 proteins in a single reaction with femtomolar detection limit and 10 logs of dynamic range (Wik et al., 2021). Somalogic developed a new aptamer-based proteomic technology for biomarker discovery capable of simultaneously measuring thousands of proteins from small sample volumes (15 μL of serum or plasma) with very low limits of detection (100 femtomolar average), 10 logs of overall dynamic range, and 5% average coefficient of variation (Gold et al., 2010; Green et al., 2001; Rohloff et al., 2014; Groote et al., 2017). Alamar Bioscience reported the development of Nucleic acid-Linked Immuno-Sandwich Assay (NULISA) with attomolar detection limit and 7-12 logs of dynamic range (Feng et al., 2023). A multiplex NULISA assay for a panel of 204 proteins, including 124 cytokines, chemokines, and other proteins involved in inflammation and immune response was able to detect previously difficult-to-detect but biologically important, low-abundance biomarkers in patients with autoimmune diseases and COVID-19.

However, these high specific, high throughput multiplex assays need to use extra molecular analytical tools or instruments to read out the detection results, such as DNA microarrays, QPCR, Mass Spectrometer and DNA sequencer etc., beside the tools or the instruments for sample preparation, target protein capturing and DNA barcode amplification, so the workflow is typically complicated and tedious. There is a strong need to integrate most steps of workflow into one instrument to simplify the assay.

### SUMMARY OF THE INVENTION

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided in some aspects are methods for detection and analysis of proteins, comprising the steps of: (a) providing a 1^{st} capture molecule on 1^{st} solid surfaces through a cleavable linker; (b) capturing a target protein with the 1^{st} capture molecule on the 1^{st} solid surfaces; (c) generating an attaching group on the captured protein without disrupting the captured protein complex; (d) cleaving the captured protein complex and immobilized on the 2^{nd} solid surfaces through the attaching group on the captured protein; (e) releasing the 1^{st} capture molecule; (f) contacting the immobilized protein with 2^{nd} capture molecule with a coding DNA tag; (g) transferring the DNA barcode in the coding DNA tag to a primer on the 2^{nd} solid surfaces; (h) optionally amplifying the extended coding DNA strand into a cluster in situ on the 2^{nd} solid surfaces; (i) decoding the DNA strands on the 2^{nd} solid surfaces.

In some embodiments, the cleavable linker in step (a) is to release the protein complex molecule from the 1^{st} solid surfaces under certain conditions, like UV illumination. The cleavable linker can be photocleavable or chemically cleavable or enzymatically cleavable.

In some embodiments, a kinetic challenge is inserted between step (c) and step (d). The kinetic challenge is performed by adding a competitor to the mixture containing the captured protein complexes and subsequently incubating the captured protein complexes in the competitor solution for a time less than or equal to the dissociation half-life of the protein affinity complexes, which can significantly increase the specificity of the detection assay. In some embodiments, the capture molecule in step (e) is released by one or more of following treatment: high salt, high pH, low pH or evaluated temperature.

In some embodiments, the assays and methods described above are used to detect and/or quantify two or more targeted proteins. Each target protein has its own specific DNA barcode encoded in a coding DNA tag. Because there are no inherent limits to detect multiple target proteins, the assay can be used to detect, for example, 2 or more target proteins, 10 or more target proteins, 25 or more target proteins, 50 or more target proteins, 100 or more target proteins, 250 or more target proteins, 500 or more target proteins, or 1000 or more target proteins.

In some embodiments, the amplification method in step (h) is rolling cycle amplification (RCA), recombinase polymerase amplification (RPA), template walking, bridge amplification, loop mediated isothermal amplification (LAMP), strand displacement amplification (SDA) and multiple displacement amplification (MDA); the decoding method in step (i) is nucleic acid hybridization assay or next generation nucleic acid sequencing.

In some embodiments, the extended DNA strand on the 2^{nd} solid surfaces in step (h) is not amplified into a cluster in situ on the 2^{nd} solid surfaces and then the decoding method in step (i) is single molecule nucleic acid hybridization assay or single molecule nucleic acid sequencing.

In some embodiments, the assays and methods described above are used to detect and/or quantify a mixture of target proteins from two and more samples. Each sample has its own specific sample ID encoded in a sample ID DNA tag. The multiplex assay comprises the steps of: (a) providing a 1^{st} capture molecule on 1^{st} solid surfaces through a cleavable linker; (b) capturing a target protein with the 1^{st} capture molecule on the 1^{st} solid surfaces; (c) attaching a sample ID DNA tag to the captured target protein without disrupting the captured protein complex; (d) cleaving the captured protein complex with a unique sample ID DNA tag; (e) pooling these protein complexes from different samples together; (f) immobilizing these complex molecules on 2^{nd} solid surface through their own sample ID DNA tags; (g) releasing the 1^{st} capture molecules; (h) contacting the immobilized proteins with 2^{nd} capture molecules with unique coding DNA tags; (i) transferring DNA barcodes in the coding DNA tags to the primers on the 2^{nd} solid surfaces; (j) optionally amplifying the sample ID strands and the extended coding DNA strands into clusters in situ on the 2^{nd} solid surfaces; (k) decoding the DNA strands on the 2^{nd} solid surfaces.

In some embodiments, the cleavable linker in step (a) is to release the protein complex molecules from the 1^{st} solid surfaces under certain conditions, like UV illumination. The cleavable linker can be photocleavable or chemically cleavable or enzymatically cleavable.

In some embodiments, a kinetic challenge is inserted between step (c) and step (d). The kinetic challenge is performed by adding a competitor to the mixture containing the captured protein complexes and subsequently incubating the capture protein complexes in the competitor solution for a time less than or equal to the dissociation half-life of the protein affinity complexes, which can significantly increase the specificity of the detection assay. In some embodiments, the capture molecules in step (g) are released by one or more of following treatment: high salt, high pH, low pH or evaluated temperature.

In some embodiments, the assays and methods described above are used to detect and/or quantify two or more target proteins from the same sample. Every target protein has its own specific DNA barcode encoded in a coding DNA tag. Because there are no inherent limits to detect multiple target proteins, the assay can be used to detect, for example, 2 or more target proteins, 10 or more target proteins, 25 or more target proteins, 50 or more target proteins, 100 or more target proteins, 250 or more target proteins, 500 or more target proteins, or 1000 or more target proteins.

In some embodiments, the assays and methods described above are also used to detect and/or quantify two or more samples. Each sample has its own specific sample ID barcode encoded in a sample ID DNA tag. Because there are no inherent limits to detect multiple samples, the assay can be used to detect, for example, 2 or more samples, 10 or more samples, 25 or more samples, 50 or more samples, 100 or more samples, 250 or more samples, 500 or more samples, or 1000 or more samples.

In some embodiments, the amplification method in step (j) is rolling cycle amplification (RCA), recombinase polymerase amplification (RPA), template walking, bridge amplification, loop mediated isothermal amplification (LAMP), strand displacement amplification (SDA) and multiple displacement amplification (MDA); The decoding method in step (k) is nucleic acid hybridization assay or next generation nucleic acid sequencing.

In some embodiments, the DNA strands in step (j) are not amplified into clusters in situ on the 2^{nd} solid surfaces and then the decoding method in step (k) is single molecule nucleic acid hybridization assay or single molecule nucleic acid sequencing.

In some embodiments, each target protein has a specific sample ID encoded in a sample ID DNA tag and a specific DNA barcode for a target protein encoded in a coding DNA tag. In some embodiments, the sample ID DNA strand is closed to the extended barcode DNA strand derived from the same target protein. The distance between the sample ID DNA tag and the extended barcode DNA strand derived from the same target protein is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances, which is hard to optically resolve detection signals from the sample ID strand and the extended barcode DNA strand derived from the same target protein. Based on this colocalization relation, each detected sample ID is correspondently assigned to the detected target protein.

Provided in other aspects is a solid support for protein detection and analysis. The solid support can be beads, silicon, glass, sapphire, or metal substrates to immobilize target proteins. The target proteins are immobilized in either random or regular array format on the solid support.

### BRIEF DESCRIPTION OF THE DRAWINGS

Non-limiting embodiments of the present invention will be described by way of example with reference to the accompanying figures, which are schematics are not intended to be drawn to scale. For purposes of illustration, not every component is labeled in every figure, nor is every component of each embodiment of the invention shown where illustration is not necessary to allow those of ordinary skill in the art to understand the invention.

FIG. 1 illustrates a detection and analysis scheme for proteins. Thus, in one embodiment, provided herein is a primer, a target protein with a sample ID DNA tag, a capture molecule with a barcode DNA tag and a reaction enzyme. The sample ID DNA tag on a target protein comprises one or more barcode sequences, for example sample ID or UMI. The barcode DNA tag on a capture molecule comprises one or more barcode sequences, for example sample ID or UMI or specific sequence for target protein etc. The barcode information encoded in a barcode DNA tag can be transferred and then extends a primer on the solid surfaces using extension assay or ligation assay. The extended primer and the sample ID strand are optionally amplified into clusters in situ on the solid surfaces. The barcode information and the sample ID information can be decoded through hybridization or sequencing. In some embodiment, the components only comprise a primer, a target protein, a capture molecule with a barcode DNA tag, and a reaction enzyme. After the barcode information is transferred to a primer on the solid surfaces, the extended primer with encoded barcode information is optionally amplified into a cluster in situ on the surfaces. The barcode information is decoded through hybridization assays or sequencing.

FIG. 2A to FIG. 2J illustrate the steps for protein detection and analysis in one embodiment. FIG. 2A illustrates that a 1^{st} capture molecule is immobilized on 1^{st} solid surfaces (e.g. magnetic beads) through a cleavable linker. FIG. 2B illustrates that the 1^{st} capture molecule binds a target protein to form an affinity complex and then the protein complex is separated from the rest of free solutions after stringent washing. FIG. 2C illustrates that a sample ID DNA tag is attached with the captured protein complex through an attaching group on the captured target protein without disrupting the captured protein complex. FIG. 2D illustrates that a target protein complex is released from the 1^{st} solid surface and then the released protein complexes from different samples are pooled together. FIG. 2E illustrates that the target protein complexes are directly immobilized on the 2^{nd} solid surfaces through a sample ID DNA tag. FIG. 2F illustrates that the 1^{st} capture molecule is released. FIG. 2G illustrates that a 2^{nd} capture molecule with a barcode DNA tag is specifically bound with an immobilized target protein. FIG. 2H illustrates that barcode DNA tag is hybridized with a primer on the 2^{nd} solid surfaces. Enzyme extends a primer and then transfers the barcode information to the primer on the 2^{nd} solid surfaces. FIG. 2I illustrates that the DNA barcode strands, including the sample ID DNA strand and the extended barcode DNA strand, are amplified into clusters in situ on the 2^{nd} solid surfaces. FIG. 2J illustrates that amplified DNA clusters are cyclically decoded using hybridization assay or sequencing.

FIG. 3 illustrates an example of capture molecule construct including a cleavable or releasable element, a tag (for example biotin), a spacer, and a capture molecule.

FIG. 4 illustrates a general overview of a sample ID DNA tag construct of a target protein, including an attaching element, a sample ID DNA tag, and a cleavable element.

FIG. 5 illustrates a general overview of a coding DNA tag construct of a capture molecule, including an attaching element, a coding DNA tag, and a cleavable element.

FIG. 6A illustrates in situ DNA strand amplification in one embodiment. The sample ID DNA strand and the extended coding DNA strand derived from the same target protein are colocalized on the 2^{nd} solid surfaces. Circular amplification primer templates are hybridized with the sample DNA tag and the extended coding DNA strand and then amplification polymerases (e.g. Phi29) follow the circular templates to incorporate nucleotides into these DNA strands. After many cycles linear duplication, these DNA strands, including the sample ID DNA strand and the extended coding DNA strand, are amplified into clusters on the 2^{nd} solid surfaces.

FIG. 6B illustrates in situ DNA strand amplification in another embodiment. The sample ID DNA strand and the extended coding DNA strand derived from the same target protein are colocalized on the 2^{nd} solid surfaces. Nicked templates are captured by the surface immobilized primers. Surface primers are extended to the full length of the template by strand displacement enzyme at 37° C. Template invasion by solution phase primers and template walk to nearby surface primer to form two copies of the template.

FIG. 7A illustrates a method of decoding DNA strands using hybridization assay. A dye labelled ID barcode probe is to decode sample information in the cluster and a dye labelled barcode probe is to decode the barcode information of the coding DNA strands colocalized on the 2^{nd} solid surfaces. An ID barcode probe is specific for a sample ID DNA strand and a barcode probe is specific for an extended coding DNA strand. Using these ID barcodes probes and the barcode probes, both the barcode information of the samples and the barcode information of the target proteins can be sequentially decoded using hybridization assays.

FIG. 7B illustrates another method of decoding DNA strands using sequencing. Both the sample ID DNA strands and the extended coding DNA strands on the 2^{nd} solid surfaces have sequencing priming sites. In some embodiments, the sample ID DNA strands and the extended coding DNA strands share the same sequencing primer. After hybridizing this primer, both the sample information of the sample ID DNA strands and the target protein information of the extended coding DNA strands can be sequentially decoded by sequencing in a stepwise method. In some embodiments, the sample ID DNA strands and the extended coding DNA strands have different sequencing primers. Thus, after hybridizing the specific primer for the sample ID DNA strands, the sample information of the sample ID DNA strands can only be sequentially decoded by sequencing in a stepwise method. After hybridizing the specific primer for the coding DNA strands, the target protein information of the extended coding DNA strands can only be sequentially decoded by sequencing in a stepwise method.

FIG. 8A to FIG. 8J illustrate the steps for highly parallelized protein detection and analysis for single sample in one embodiment. FIG. 8A illustrates that a 1^{st} capture molecule is immobilized on 1^{st} solid surfaces (e.g. magnetic beads) through a cleavable linker. FIG. 8B illustrates that the 1^{st} capture molecule binds a target protein to form a protein affinity complex and then the protein affinity complex is separated from the rest of the solution after stringent washing. FIG. 8C illustrates that an attaching group is generated on the captured protein without disrupting the captured protein complex. FIG. 8D illustrates that the target protein complex is released. FIG. 8E illustrates that the target protein complex is directly immobilized on 2^{nd} solid surface through the attaching group on the captured protein. FIG. 8F illustrates that the 1^{st} capture molecule is released. FIG. 8G illustrates that the a 2^{nd} capture molecule with a barcode DNA tag is specifically bound with the target protein. FIG. 8H illustrates that the barcode DNA tag is hybridized with a primer on the 2^{nd} solid surfaces. Enzyme extends a primer and then transfer the barcode information to the primer on the 2^{nd} solid surfaces. FIG. 8I illustrates that the extended DNA strand with barcode information is amplified into a cluster in situ on the 2^{nd} solid surfaces. FIG. 8J illustrates that the amplified DNA cluster is cyclically decoded using hybridization assay or sequencing.

### DETAILED DESCRIPTION OF THE PREFERRED EMBODIMENTS

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an” and “the” include plural references unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

As used herein, the term “detection” is used to broadly to include any means of determining the presence of the analyte or any form of measurement of the analyte. Thus, “detecting” can include determining, measuring, or assessing the presence or absence or amount or location of analyte. Quantitative, semi-quantitative and qualitative determinations, measurements or assessments are included. Such determinations, measurements or assessments can be relative, for example, when two or more different analytes in a sample are being detected.

As used herein, the term “sample” can be any biological and clinical samples, included, e.g. any cell or tissue sample of an organism, or any body fluid or preparation derived therefrom, as well as sample such as cell cultures, cell preparations, cell lysates, etc. Environmental sample, e.g. soil and water samples or food samples are also included. The samples can be freshly prepared or prior-treated in any convenient way. Representative samples thus include any material that contains a biomolecule, or any other desired or targeted analyte, including, for example, foods or allied products, clinical and environmental samples. The sample can be a biological sample, including viral or cellular materials, including prokaryotic or eukaryotic cells, viruses, bacteriophages, mycoplasmas, protoplasts, and organelles. Such biological material comprises all types of mammalian and non-mammalian animal cells, plant cells, algae. Representative samples also include whole blood and blood-derived product such as plasma, serum and buffy coat, blood cells, urine, faeces, cerebrospinal fluid or any other body fluids (e.g. respiratory secretion, saliva, milk, etc.), tissues, biopsies, cell cultures, cell suspensions, conditioned media or other samples of cell culture constituents, etc. The sample can be pre-treated in any convenient or desired way to prepare for use in the method disclosed herein. For example, the sample can be treated by cell lysis or purification, isolation of the analyte, etc.

As used herein, the term “proteome” can include the entire set of proteins, polypeptides, or peptides expressed by a genome, cell, tissue, or organism at a certain time. It is the set of expressed proteins in a given type of cell or organism, at a given time, under defined conditions. A cellular proteome is the collection of proteins found in a particular cell type under a particular set of environmental conditions. In some aspect, proteome refers to the collection of proteins in certain sub-cellular systems, such as organelles. As used herein, the term “proteomics” refers to quantitative analysis of the proteome within cells, tissues, and body fluids and the corresponding spatial distribution of the proteome within the cell and within tissues.

As used herein, the term “capture molecule”, “probe” or “receptor” refers to a molecule that is configured to associate, either directly or indirectly, with a tag. A “capture molecule”, “probe” or “receptor” is a set of copies of one type of molecule or one type of multi-molecular structure that is capable of immobilizing the moiety to which the tag is attached to a solid support by associating, either directly or indirectly, with the tag. A capture molecule, probe or receptor can be a polynucleotide, a polypeptide, a peptide nucleic acid, a locked nucleic acid, an oligosaccharide, a polysaccharide, an antibody, an affibody, an antibody mimic, a cell receptor, a ligand, a lipid, biotin, polyhistidine, or any fragment or derivatives of these structures, any combination of the foregoing, or any other structures with which a tag (or linker molecule) can be designed or configured to bind or otherwise associate with specificity. A capture molecule, probe, o receptor can be attached to a solid support either covalently or non-covalently by any suitable method.

As used herein, the term “binding” refers to an interaction between molecules (e.g. a binding molecule and an analyte, or a presenting group and a receiving group) to form a complex. Interactions can be, for example, non-covalent interaction including hydrogen bonds, ionic bonds, hydrophobic interactions, and/or van der Waals interactions. A “binding molecule”, as used herein in connection with an analyte, is any molecule or entity capable of binding to the analyte. In some embodiments, the binding molecules binds to the target analyte with greater affinity than to other components in the sample. In some embodiments, the binding molecule's binding to the target analyte can be distinguished from that to non-target analytes or does so negligibly or non-detectably, or any such non-specific binding, if it occurs, is at a relatively low level that can be distinguished. The binding between the target analyte and its binding molecule is typically non-covalent. The binding molecule used in the methods provided herein can be covalently conjugated to a presenting group (e.g. a nucleic acid tag) without substantially abolishing the binding affinity of the binding molecule to its target analyte.

As used herein, the term “protein complex” or “protein affinity complex” refers to a non-covalent complex that is formed by the interaction of a binding or capture molecule with its targeted molecule. A “protein complex” or “protein affinity complex” is a set of copies of one type of species of complex formed by a binding or capture molecule bound to its corresponding target molecule. A protein affinity complex or protein complex can generally be reversed or dissociated by a change in an environmental condition, e.g., an increase in temperature, an increase in salt concentration, or an addition of a denaturant.

As used herein, the term “partition” refers to a separation or removal of one or more molecular species from the test sample. Partitioning can be used to increase sensitivity and/or reduce background. Partitioning is most effective following protein complex formation. A partitioning step may be introduced after any step, or after every step, where the protein affinity complex is immobilized. Partitioning may also rely on a size differential or other specific property that differentially exists between the protein affinity complex and other components of the test sample. Partitioning may also be achieved through a specific interaction with a protein, a capture molecule and protein affinity complex.

As used herein, the term “cleavable linker” or “cleavable element” refers to a group of atoms that contains a releasable or cleavable element. In some embodiments, a cleavable linker is used to join a protein to a tag, thereby forming a releasable tag. For example, a releasable linker can be utilized in any of the described assays to create a releasable connection between a protein and a biotin. In one embodiment, the cleavable linker may be photocleavable in that it includes a bond that can be cleaved by irradiating the releasable element at the appropriate wavelength of light. In another embodiment, the cleavable linker may be chemically cleavable in that it includes a bond that can be cleaved by treating it with an appropriate chemical or enzymatic reagent. In another embodiment, the releasable element includes a disulfide bond that can be cleaved by treating it with a reducing agent to disrupt the bond.

As used herein, the term “competitor molecule” and “competitor” are used interchangeably to refer to any molecule that can form a non-specific complex with a non-target molecule, for example to prevent that non-target molecule form rebinding non-specifically to a binding molecule. A “competitor molecule” or “competitor” is a set of copies of one type or species of molecule. Competitor molecules include oligonucleotides, polyanions, abasic phosphodiester polymers, dNTPs, and pyrophosphate. In the case of a kinetic challenge that use a competitor, the competitor can also be any molecule that can form a non-specific complex with a free capture molecule, for example to prevent that the capture molecule from rebinding non-specifically to a non-target protein.

As used herein, the term “barcode” refers to a unique sequence associated with a polynucleotide. This chain may have 2 to about 30 bases of nucleic acid units and the unique nucleic acid sequence provides an identification or origin information for a target protein or reaction cycle, or a set of samples etc. In certain embodiments, each barcode within a population of barcodes is different. Barcode can be computationally deconvoluted derived from an individual protein, sample, library, etc. A barcode can also be used for deconvolution of collection of proteins or nucleotides that have been distributed into small compartments for enhanced mapping.

As used herein, the term “sample ID” refers to a barcode that identifies from which sample a target protein derives or come from.

As used herein, the term “coding tag”, “coding DNA tag” or “barcode DNA tag” refers to a polynucleotide with unique sequence identifying information for its associated chemical agent. A coding tag may also be comprised of an optional UMI and/or an optional reaction cycle-specific barcode. In certain embodiments, a coding tag may further comprise a reaction cycle specific barcode, a unique molecular identifier, a universal primming site, or any combination thereof.

As used herein, the term “primer” refers to a polynucleotide molecule, which may be used for library amplification, extension, ligation and/or for sequencing reactions. In some aspect, a primer can be used for amplification. For example, extended DNA strands from a primer can be used for rolling circle amplification to form DNA nanoballs that can be used as sequencing templates. Alternatively, extended DNA strands may be amplificated into clusters in situ and then sequenced by polymerase extension from primers.

As used herein, the term “solid support” or “solid surfaces” or “substrate” refers to any solid materials to which a protein can be attached directly or indirectly by covalent or non-covalent interactions, or any combination thereof. A solid support may be two-dimensional planar surface or three-dimensional surface. A solid support can be any support surface including, but not limited to, a bead, a microbead, an array, a glass surface, a silicon surface, a plastic surface etc. Materials for a solid support include but are not limited to acrylamide, agarose, cellulose, glass, gold, quartz, polystryrene, polyethylene, plyethylene oxide, polysilicates, polycarbonates, Teflon, fluorocarbons, nylon, functionalized silane, collagen, polyamino acids, or any combination thereof. Solid supports further include thin film, membrane, polymers such as particles, beads, microspheres, microparticles, or any combination thereof.

As used herein, the term “sequencing” refers to the technique for the determination of the order of molecules, such as nucleotides or amino acids, in a ligand molecule, such as polynucleotide or polypeptide, or a sample of ligand molecules.

As used herein, the term “next generation sequencing” refers to high throughput sequencing methods that allow the sequencing of millions to billions of molecules in parallel. Examples of next generation sequencing methods include sequencing by synthesis, sequencing by ligation, sequencing by hybridization, semiconductor sequencing, and pyrosequencing. By attaching primers to a solid surface and a complementary sequence to a nucleic acid molecule, a nucleic acid molecule can be hybridized to the solid surfaces via the primer and then multiple copies can be generated in a discrete area on the solid surface by using polymerase to amplify. Consequently, during the sequencing process, a nucleotide at a particular position can be sequenced multiple times, which is referred to as depth of sequencing. Examples of high throughput nucleic acid sequencing technology include platforms provided by Illumina, MGI, Qiagen, Thermo Fisher, Genemind, and Roche.

As used herein, the term “single molecule sequencing” refers to the sequencing method wherein reads from single molecule are generated by sequencing of a single molecule of DNA. Unlike next generation sequencing methods that rely on amplification to clone many DNA molecules in parallel for sequencing in a stepwise approach, single molecule sequencing interrogates single molecules of DNA and does not require amplification. Examples of single molecule methods include single molecule real time sequencing (Pacific Biosciences), nanopore based sequencing (Oxford Nanopore), single molecule stepwise sequencing (Helicos Biosciences).

### Methods of Protein Detection and Analysis

Provided in some aspects are methods for protein detection and analysis. The methods described herein provide a highly parallelized and highly multiplex approach for protein detection and analysis. In some embodiments, the method described herein provide a highly parallelized approach for protein detection and analysis.

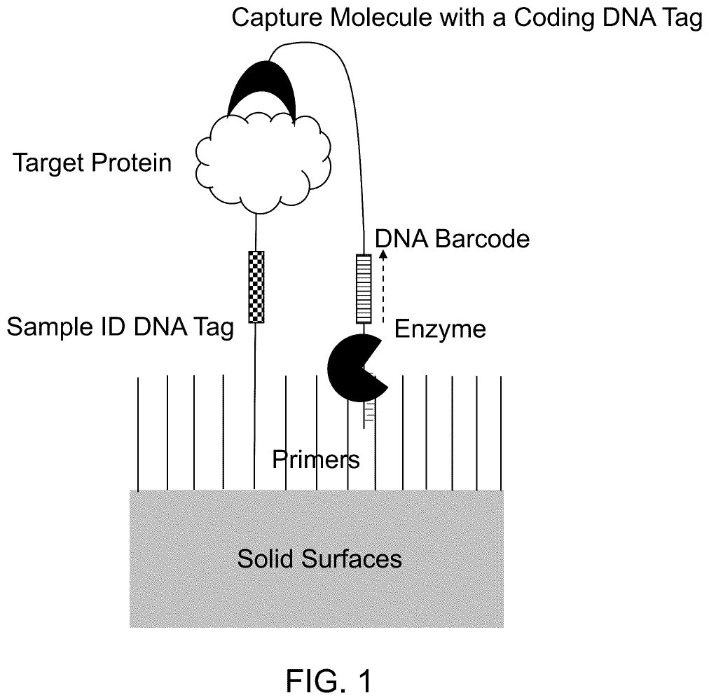

FIG. 1 illustrates a detection and analysis scheme for proteins. A target protein is directly immobilized on solid surfaces through a sample ID DNA tag. A capture molecule with a barcode DNA tag is specifically bound with the target protein. The barcode DNA tag is hybridized with a primer on the solid surfaces. Enzyme extends the primer and transfers barcode information to the primer to form an extended coding DNA strand on the solid surfaces. The extended coding DNA strand and the sample ID strand are optionally amplified into clusters in situ on the solid surfaces. The barcode information of the target proteins and the sample ID information can be decoded through hybridization assays or sequencing.

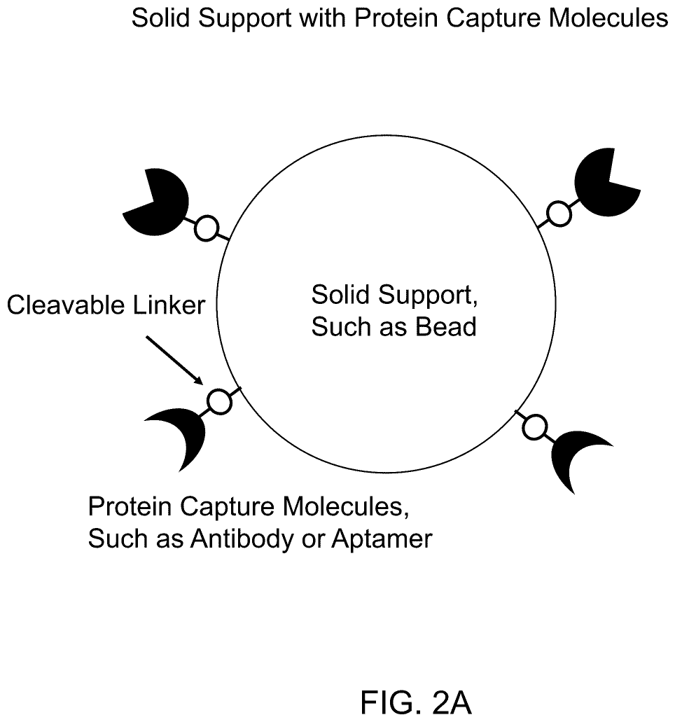

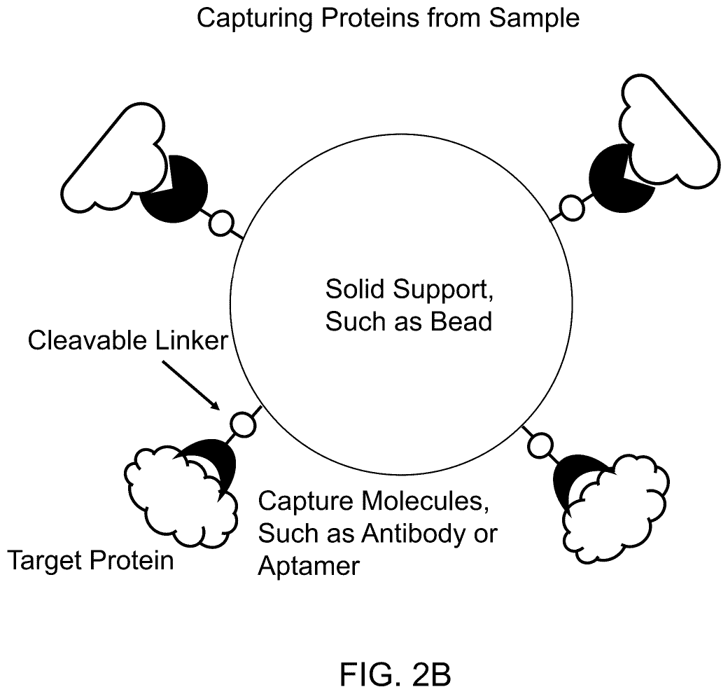

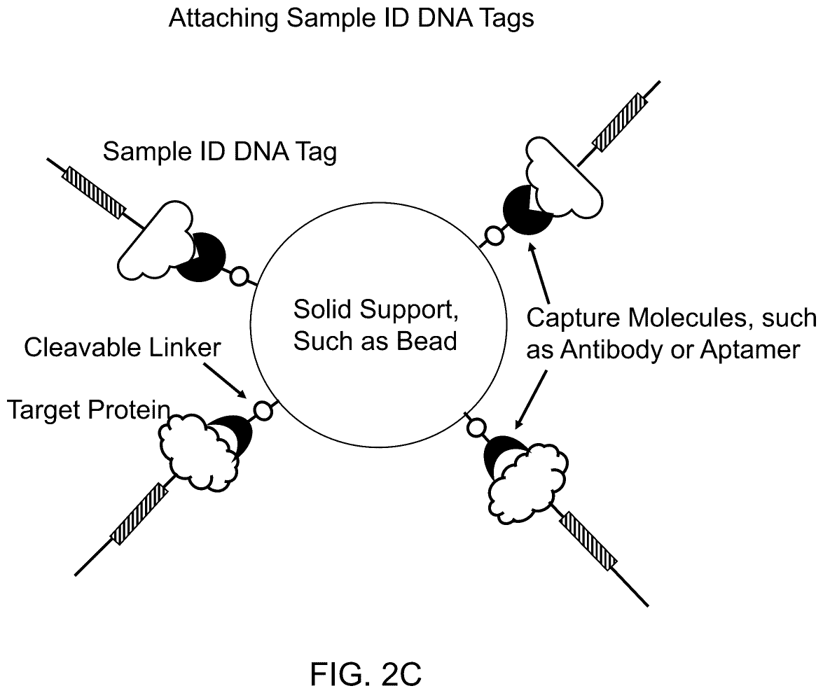

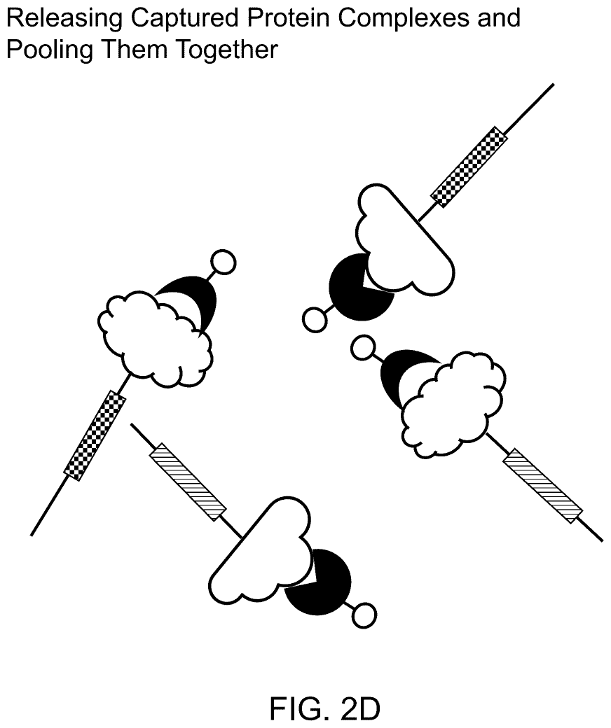

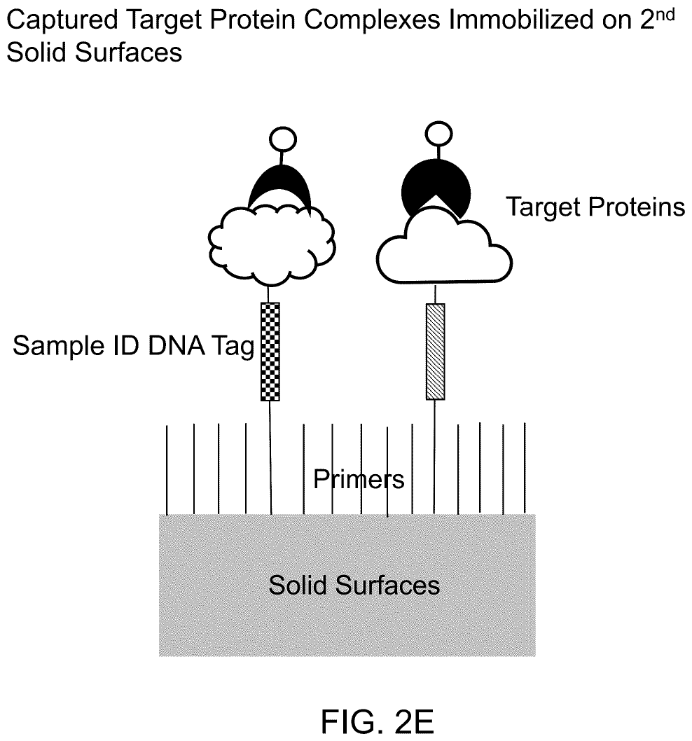

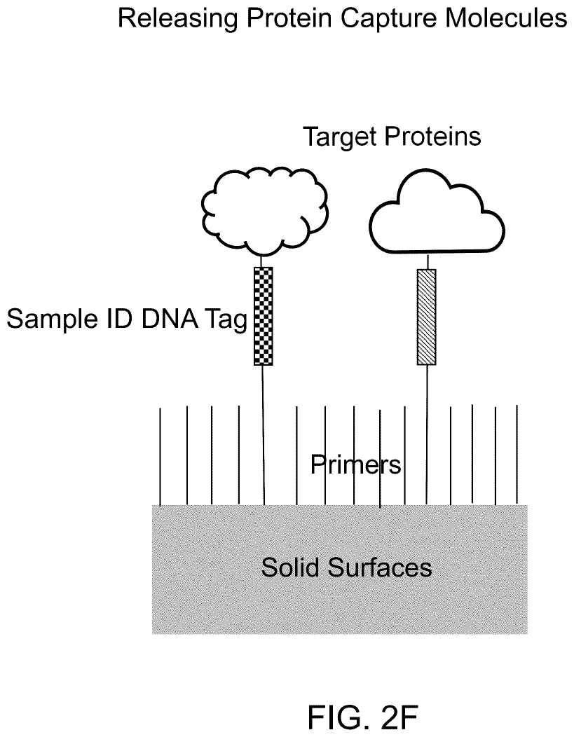

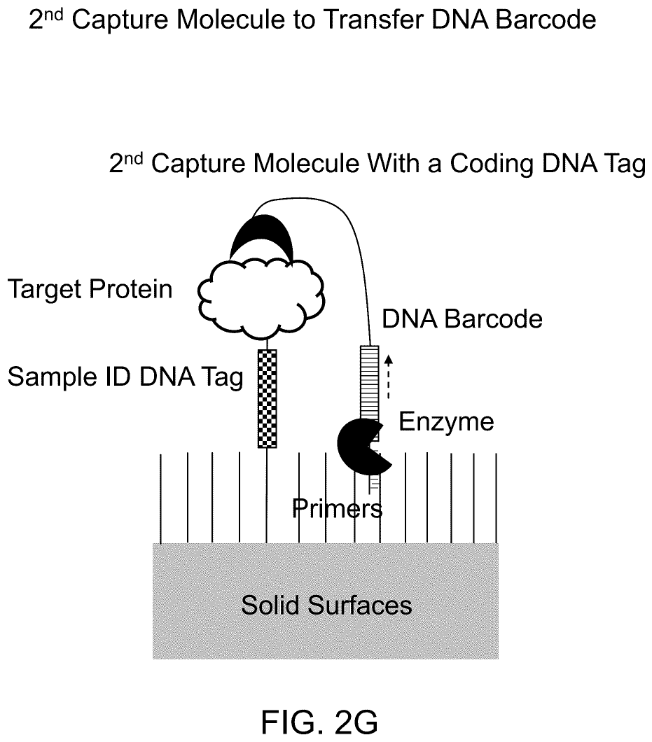

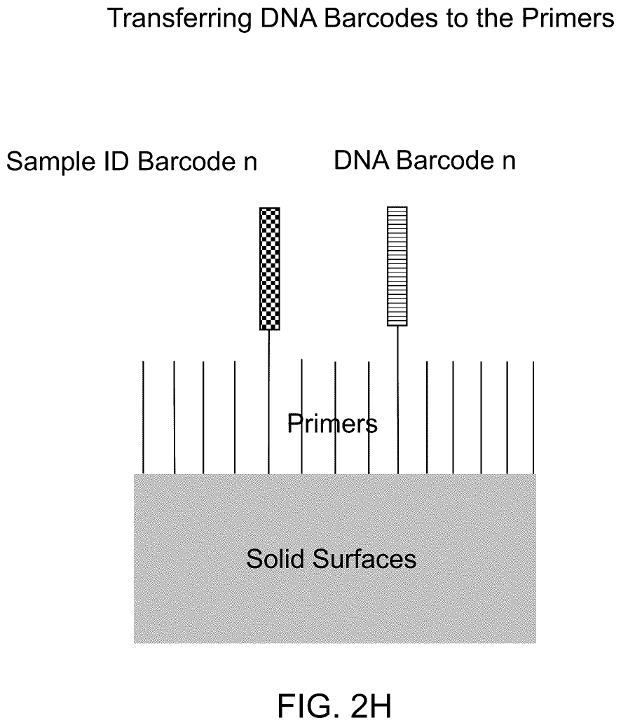

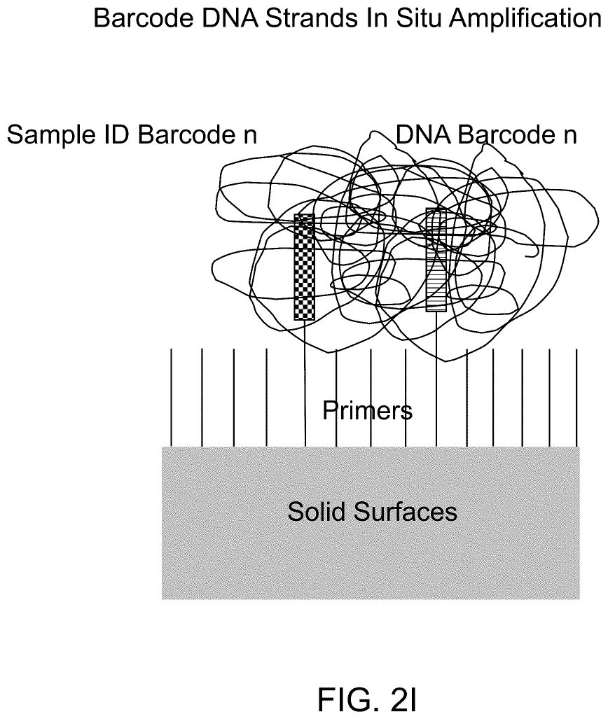

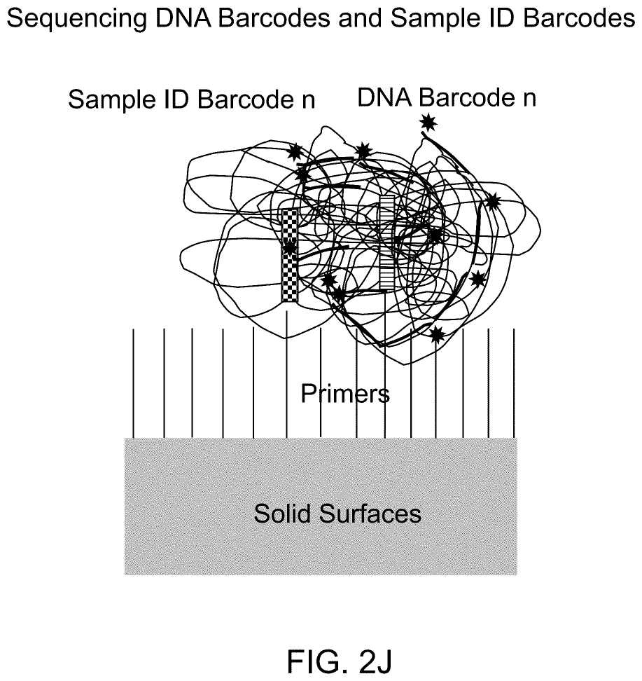

FIG. 2A to FIG. 2J illustrates an assay or a method to detect and/or quantify the targeted proteins from two and more samples. The multiplex assay comprises the steps of: (a) providing a 1^{st} capture molecule on 1^{st} solid surfaces through a cleavable linker; (b) capturing a target protein with the 1^{st} capture molecule on the 1^{st} solid surfaces; (c) attaching a sample ID DNA tag with the captured protein without disrupting the captured protein complex; (d) cleaving the captured protein complex with a unique sample ID DNA tag; (e) pooling protein complexes from different samples together; (f) immobilizing these complex molecules on the 2^{nd} solid surface through the sample ID DNA tags; (g) releasing the 1^{st} capture molecules; (h) contacting the immobilized protein complexes with 2^{nd} capture molecules with unique coding DNA tags; (i) transferring DNA barcodes to the primers the 2^{nd} solid surfaces; (j) optionally amplifying the sample ID strands and the extended coding DNA strands into clusters in situ on the 2^{nd} solid surfaces (k) decoding these DNA strands on the 2^{nd} solid surfaces.

In some embodiments, the 1^{st} capture molecule is immobilized on the 1^{st} solid surface through a cleavable linker (FIG. 2A). A solid surface can be two-dimensional planar surface or three-dimensional surface, including, but not limited to, a bead, a microbead, an array, a glass surface, a silicon surface, a plastic surface etc. Materials for a solid surface include but are not limited to acrylamide, agarose, cellulose, glass, gold, quartz, polystryrene, polyethylene, plyethylene oxide, polysilicates, polycarbonates, Teflon, fluorocarbons, nylon, functionalized silane, collagen, polyamine acids, or any combination thereof. The cleavable linker has an attaching group that includes, but is not limited to, primary amine, carboxylic acid, alkyne, acryloyl, allyl and aldehyde. The surface functional groups on the 1^{st} solid surface can covalently immobilize the protein capture molecules on the surfaces through the attaching group of a cleavable linker. The surface functional groups include, but not limited to, aldehyde, oxime, hydrazone, hydrazide, alkyne, amine, azide, acylazide, acylhalide, nitrile, nitrone, sulfhydryl, disulfide, sulfonyl halide, isothiocyanate, imidoester, N-hydroxysuccinimide ester, pentynoic acid ester. In some embodiments, the cleavable linker is immobilized on the 1^{st} solid surface through an affinity pair. The affinity pair includes, but not limited to, an antigen and an antibody against the antigen (including its fragments, derivatives, or mimetics), a ligand and its receptor, complementary strands of nucleic acids (e.g., Poly A or Ploy T), biotin and avidin (or streptavidin or neutravidin), lectin and carbohydrates, and vice versa.

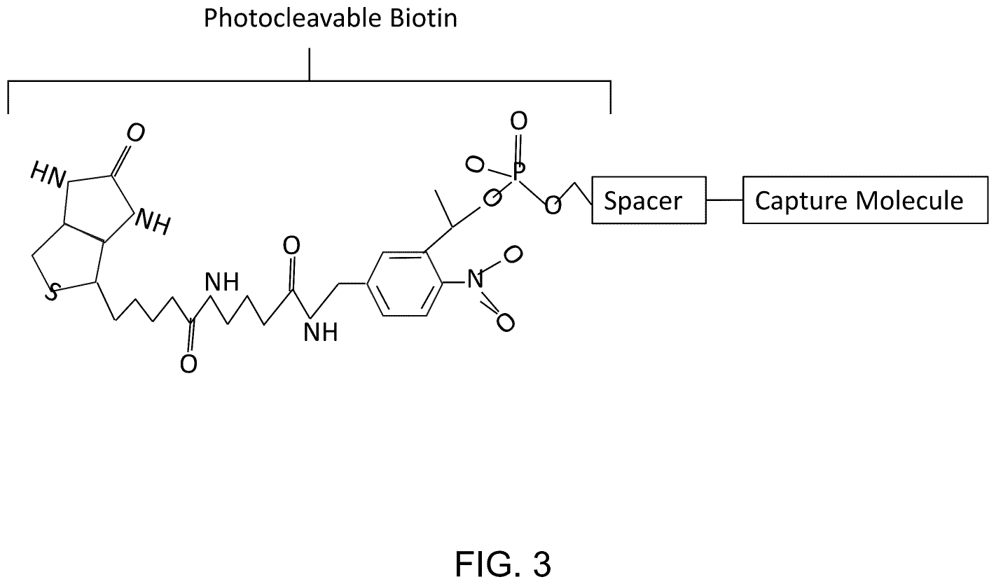

The 1^{st} capture molecule has a cleavable linker, which can release the captured protein complex from the 1^{st} solid surfaces. In one embodiment, the cleavable linker may be photocleavable in that it includes a bond that can be cleaved by irradiating the releasable element at the appropriate wavelength of light. For example, to cleave the photocleavable linker, the mixture is irradiated with a UV lamp for about 20 minutes. In some embodiments, the cleavable linker may be chemically cleavable in that it includes a bond that can be cleaved by treating it with an appropriate chemical or enzymatic reagent. In another embodiment, the releasable element includes a disulfide bond that can be cleaved by treating it with a reducing agent to disrupt the bond. FIG. 3 illustrates an example of the 1^{st} capture molecule construct including a cleavable or releasable element, a tag (for example biotin), a spacer and a capture molecule.

With the reference to FIG. 2B, the presence of target proteins in a test sample is detected and/or captured by contacting a test sample with the 1^{st} capture molecules on 1^{st} solid surfaces (e.g. magnetic beads) that have specific affinity for these target proteins. The mixture may optionally be incubated for a period of time sufficient to achieve equilibrium binding of the capture molecules and the target proteins (e.g. at least 10 minutes, at the least about 20 minutes, at the least about 30 minutes). A partition step is then employed to remove the rest of free molecules inside solution to eliminate the potential noise in the assay.

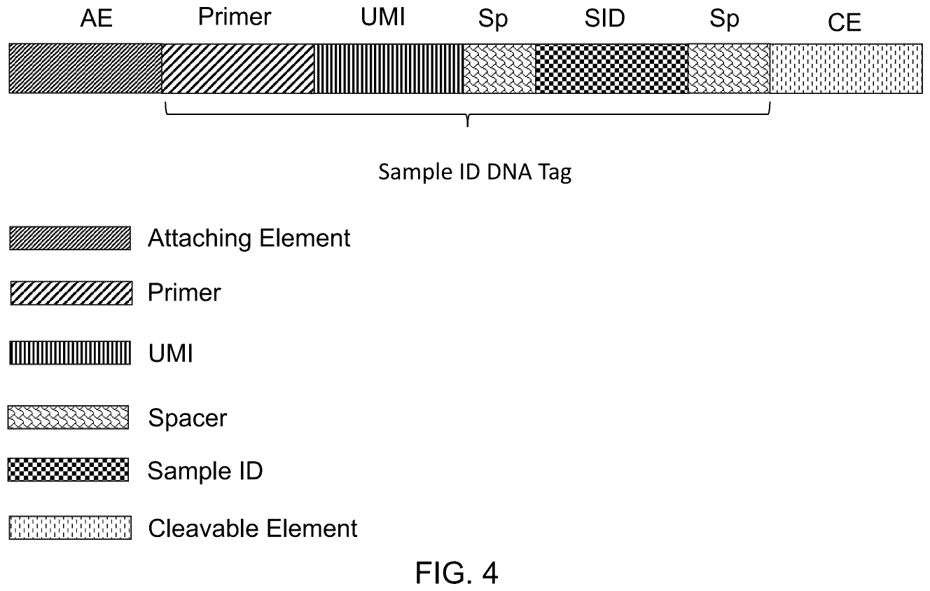

With reference to FIG. 2C, the capture protein has a free attaching group that can be used to attach a sample ID DNA tag. The sample ID DNA tag has unique sequence information for different samples. Provided herein is a sample ID DNA tag, comprising one or more primer sequences, one or more unique molecular identifier (UMI) sequences, one or more spacer sequences, one or more sample ID sequence, one or more compartment sequences, one or more sequencing cycle number sequences or any combination thereof. In some embodiments, the sample ID DNA tag connects an attaching element or a cleavable element or combination of these two elements. In some embodiments, the attaching element is a primer or a complementary primer. In some embodiments, the attachment element is a functional group that can covalently attached on the solid surfaces. In some embodiments, the attachment element is an affinity pair. The affinity pair includes, but not limited to, an antigen and an antibody against the antigen (including its fragments, derivatives, or mimetics), a ligand and its receptor, complementary strands of nucleic acids (e.g., Poly A or Ploy T), biotin and avidin (or streptavidin or neutravidin), lectin and carbohydrates, and vice versa. FIG. 4 illustrates a general overview of a sample ID DNA tag construct, including an attaching element, a sample ID DNA tag, and a cleavable element.

In certain embodiments, a unique molecular identifier (UMI) provides a unique identifier tag for each sample to which the UMI is associated with. A UMI can be about 3 to about 40 bases, about 3 to about 30 bases, about 3 to about 20 bases, about 3 to about 10 bases, about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases or any number of bases between two aforementioned numbers of bases in length.

A kinetic challenge is optionally performed to remove or reduce the non-specific protein affinity complexes to improve the specificity of the assay. The kinetic challenge is performed by adding a competitor to the mixture containing the captured protein complexes and subsequently incubating the capture protein complexes in the competitor solution for a time less than or equal to the dissociation half-life of the protein affinity complexes, which can significantly increase the specificity of the detection assay.

With reference to FIG. 2D, the capture protein complexes can be released by disrupting the cleavable linker under certain conditions. In some embodiments, the cleavable linker is a photocleavable linker that is cleaved by irradiation with a UV lamp under conditions that can cleave >90% of the linker. In another embodiment, the cleavable linker may be chemically cleavable in that it includes a bond that can be cleaved by treating it with an appropriate chemical or enzymatic reagent. In another embodiment, the releasable element includes a disulfide bond that can be cleaved by treating it with a reducing agent to disrupt the bond. The released protein complex has a unique sample ID DNA tag and can be pooled together for a highly parallelized and highly multiplex assay. Because there are no inherent limits to detect multiple targets from the same sample, the assay can be used to detect, for example, 2 or more targets, 10 or more targets, 25 or more targets, 50 or more targets, 100 or more targets, 250 or more targets, 500 or more targets, or 1000 or more targets. Moreover, there are also no inherent limits to detect multiple samples, the assay can be used to detect, for example, 2 or more samples, 10 or more samples, 25 or more samples, 50 or more samples, 100 or more samples, 250 or more samples, 500 or more samples, or 1000 or more samples.

With the reference to FIG. 2E, a pool of the released protein complexes is directly immobilized on the 2^{nd} solid surfaces through sample ID DNA tags. The 2^{nd} solid surface can be two-dimensional planar surface or three-dimensional surface, including, but not limited to, a bead, a microbead, an array, a glass surface, a silicon surface, a plastic surface etc. Materials for a solid surface include but are not limited to acrylamide, agarose, cellulose, glass, gold, quartz, polystryrene, polyethylene, plyethylene oxide, polysilicates, polycarbonates, Teflon, fluorocarbons, nylon, functionalized silane, collagen, polyamine acids, or any combination thereof. The sample ID DNA tag has an attaching group that includes, but is not limited to, primary amine, carboxylic acid, alkyne, acryloyl, allyl and aldehyde. The functional groups on the 2^{nd} solid surface can covalently immobilize the protein complexes on the surfaces through the attaching group of the sample ID DNA tag. The surface functional groups include, but not limited to, aldehyde, oxime, hydrazone, hydrazide, alkyne, amine, azide, acylazide, acylhalide, nitrile, nitrone, sulfhydryl, disulfide, sulfonyl halide, isothiocyanate, imidoester, N-hydroxysuccinimide ester, pentynoic acid ester. In some embodiments, the protein complexes are immobilized on the 2^{nd} solid surface through an affinity pair formed by an affinity molecule of the sample ID DNA tag and another correspondent affinity molecule on the 2^{nd} solid surface. The affinity pair includes, but not limited to, an antigen and an antibody against the antigen (including its fragments, derivatives, or mimetics), a ligand and its receptor, complementary strands of nucleic acids (e.g., Poly A or Ploy T), biotin and avidin (or streptavidin or neutravidin), lectin and carbohydrates, and vice versa.

With the reference to FIG. 2F, the 1^{st} capture molecules on the protein complexes immobilized on the 2^{nd} solid surfaces can be released by one or more of following treatment: high salt, high pH, low pH or evaluated temperature.

With the reference to FIG. 2G, a pool of 2^{nd} capture molecules contacts the immobilized proteins on the 2^{nd} solid surfaces to form new protein affinity complexes again. In some embodiments, the 2^{nd} capture molecule described also comprises a coding DNA tag containing identifying information regarding associated proteins. A coding tag is a nucleic acid molecule of about 3 bases to about 100 bases that provides unique identifying information for its associated target protein. A coding DNA tag may comprise about 3 to about 90 bases, about 3 to about 80 bases, about 3 to about 70 bases, about 3 to about 60 bases, about 3 to about 50 bases, about 3 to about 40 bases, about 3 to about 30 bases, about 3 to about 20 bases, about 3 to about 10 bases, about 3 to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 50 bases, 60 bases, 70 bases, 80 bases, 90 bases, 100 bases., 200 bases, 300 bases, 400 bases, 500 bases or any number of bases between two aforementioned number of bases. A coding tag may be composed of DNA, RNA, polynucleotide analogs, or a combination thereof.

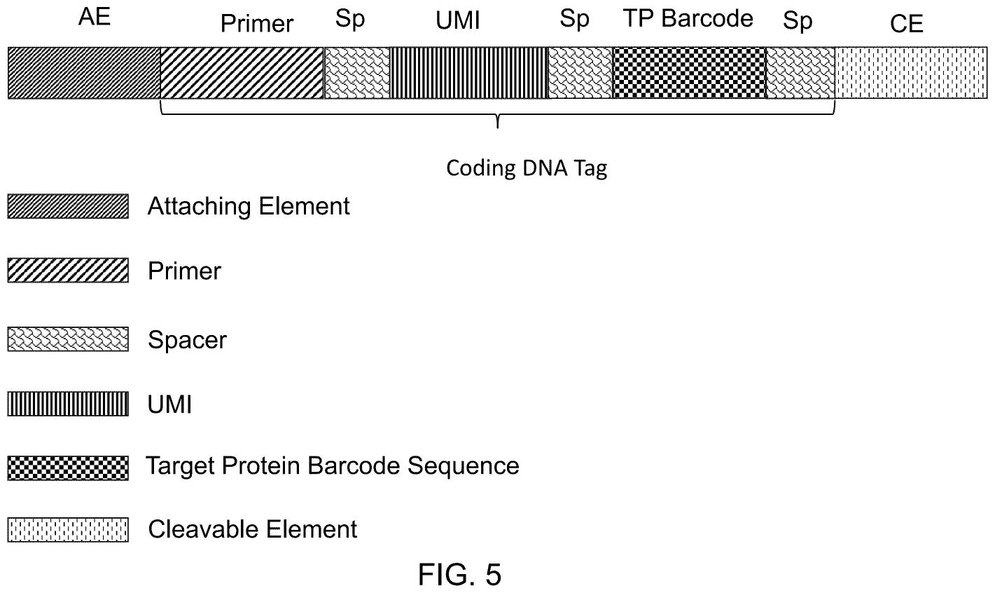

In some embodiments, the coding DNA tag of the 2^{nd} capture molecule comprises one or more primer sequences, one or more unique molecular identifier (UMI) sequences, one or more spacer sequences, one or more sample ID sequence, one or more compartment sequences, one or more sequencing cycle number sequences or any combination thereof. In some embodiments, the coding DNA tag of the 2^{nd} capture molecule connects an attaching element or a cleavable element or combination of these two elements. In some embodiments, the attaching element is a primer or a complementary primer. FIG. 5 illustrates a general overview of a coding DNA tag construct of a 2^{nd} capture molecule, including an attaching element, a coding DNA tag, and a cleavable element.

In some embodiments, the information transfer from the DNA barcode of the 2^{nd} capture molecules to the primers on the solid surfaces can be accomplished using a primer extension step. A sequence on the 3″ terminus of a primer on the solid surfaces anneals with complementary sequence on the 3′ terminus of a coding DNA tag of the 2^{nd} capture molecule and a polymerase extends the primer sequence using the annealed coding DNA tag as the template. Examples of such polymerases includes, but not limited to, Klenow, T4 DNA polymerase, T7 DNA polymerase, Bst DNA polymerase, Bca Pol, 9⁰N Pol and Phi 29 Pol.

In some embodiments, the information transfer from DNA barcode of the 2^{nd} capture molecules to the primers on the solid surfaces can be accomplished using a ligation step. Ligation may be an enzymatic ligation reaction. Examples of ligase include, but not limited to, T3 DNA ligase, T4 DNA ligase, T7 DNA ligase, Taq DNA ligase, E. coli DNA ligase, 9⁰N DNA ligase, Electroligase etc. Alternatively, a ligation may be a chemical ligation reaction (Gunderson, Huang et al., 1998, El-Sgaheer, Cheong et al. 2011).

The extended coding DNA strand is colocalized with the associated target protein on the surfaces (FIG. 2H). The distance between the extended coding DNA strand and the associated target protein is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

In some embodiments. the primer on the solid surfaces comprises a universal priming site. The universal priming site is a nucleic acid sequence that may be used for priming a library amplification reaction and/or for sequencing. A primer may include, but is not limited to, a priming site for amplification, adaptor sequence that anneal to complementary oligonucleotides on a coding DNA tag of a 2^{nd} capture molecule, a sequencing priming site, or a combination thereof. A primer can be about 10 bases to about 60 bases. In some embodiments, the primer has low melting temperature property (e.g. Poly A or Ploy T in Ma et al., PNAS, 2013, 110(35), 14320-14323). The low melting temperature may include, but is not limited to, 25° C., 30° C., 35° C., 40° C., 45° C., 50° C., 55° C., 60° C. and above, or any temperature between two aforementioned temperatures. When a 2^{nd} capture molecule binds with a target protein at an elevated temperature above the melting temperature of the primer, the barcode DNA tags on the 2^{nd} capture molecule cannot hybridize with the primer on the solid surfaces. After the capture molecule specifically binds with a target protein, the reaction temperature drops a room temperature or any temperature below the melting temperature of the primer, the barcode DNA tags on the 2^{nd} capture molecule would hybridize with the primer on the solid surfaces and then DNA barcode information transfer processes can start.

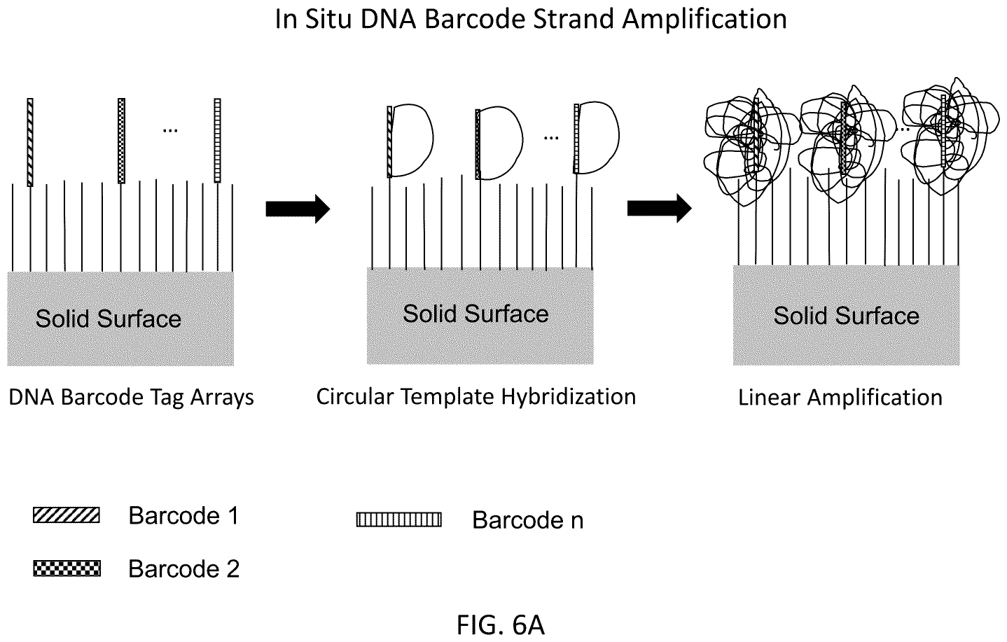

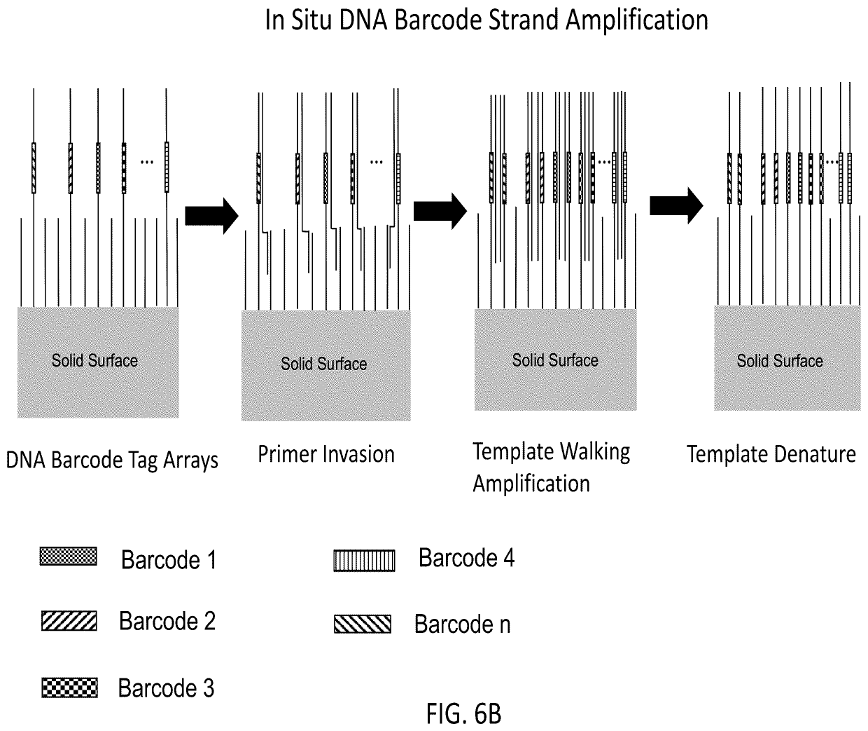

With the reference to FIG. 2I, all of DNA strands, including the extended coding DNA strands and the sample ID DNA strands, are amplified into clusters in situ on the 2^{nd} solid surfaces. In some embodiments, exemplary DNA barcode amplification method in present disclosure may be isothermal amplification such as rolling cycle amplification (RCA). RCA is an isothermal DNA amplification method that can rapidly synthesize multiple copies of circular molecules of DNA. RCA is initiated by an initiator protein encoded by the plasmid or bacteriophage DNA, which nicks one strand of the double stranded, circular DNA molecule at a stie called the double strand origin. The initiator protein remains bound to the 5′ phosphate end of the nicked strand and the free 3′ hydroxyl end is released to serve as a primer for DNA synthesis by DNA polymerase. Using the unnicked strand as a template, amplification proceeds around the circular DNA molecule, displacing the nicked strand as single stranded DNA. Continued DNA amplification can produce multiple single stranded linear copies of the original circular DNA template (FIG. 6A).

Another exemplary DNA barcode amplification method is template walking amplification (Ma et al., PNAS, 2013). Nicked templates are captured by the surface immobilized primers. Surface primers are extended to the full length of the template by strand displacement enzyme at 37° C. Template invasion by solution phase primers and template walk to nearby surface primer to form two copies of the template. The process cycle repeats to replicate a few thousand copies of the template clusters at 60° C. and 30 mins at single spot (FIG. 6B). The detail processes are described in U.S. patent Ser. No. 10/233,488 B2, which is incorporated herein by reference in its entirety.

Another exemplary molecule amplification method in present disclosure may be isothermal amplification such as recombinase polymerase amplification (RPA). The RPA reaction exploits enzymes known as recombinases, which form complexes with oligonucleotide primers and pair the primers with their homologous sequences in duplex DNA. A single-stranded DNA binding (SSB) protein binds to the displaced DNA strand and stabilizes the resulting D loop. DNA amplification by polymerase is then initiated from the primer, but only if the target sequence is present. Once initiated, the amplification reaction progresses rapidly, so that starting with just single copy of DNA or molecule, the highly specific DNA amplification reaches detectable levels within minutes at 37-42° C.

Other useful methods for amplifying nucleic acids are bridge amplification, loop mediated isothermal amplification (LAMP), strand displacement amplification (SDA) and multiple displacement amplification (MDA).

In some embodiments, the sample ID DNA strand and the extended coding DNA strand derived from the same target protein are colocalized on the solid surfaces. The distance between the sample ID DNA strand and the extended coding DNA strand derived from the same protein is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances. After the amplification, these amplified clusters derived from the same protein are also colocalized on the surfaces. The distance between these clusters amplified from the sample ID DNA strand and the extended coding DNA strand derived from the same target protein, is less than 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances, which is hard to optically resolved the detection signals from the sample ID strands and the detection signals from the extended coding DNA strands. Based on this colocalization relation, each detected sample ID is correspondently assigned to a detected target protein.

With the reference FIG. 2J, these amplified DNA strands are decoded to get the information of target proteins and the information of samples. In some embodiments, DNA barcode is cyclically decoded by primer hybridization assay using dye labeled detection probes. There are five types of nucleic acid hybridization assay: sandwich hybridization assay, competitive hybridization assay, hybridization ligation assay, dual ligation hybridization assay (DLA) and nuclease hybridization assay. In the sandwich hybridization ELISA assay format, complementary oligonucleotide capture hybridizes with a nucleic acid analyte and a labeled detection probe hybridizes with the nucleic acid analyte to form the sandwich format for detection. The completive hybridization assay relies on complementarity, where the capture probe competes between the analyte and the tracer, a labelled oligonucleotide analog the analyte. In the hybridization ligation assay, a template probe replaces the capture probe in the sandwich assay for immobilization to the solid support. The template probe is fully complementary to the oligonucleotide analyte and is intended to serve as a substrate for T4 DNA ligase-mediated ligation. The template probe has an additional stretch complementary to a ligation probe so that the ligation probe will ligate onto the 3′ end of the analyte. The ligation probe is similar to a detection probe in that it is labelled for detection. The dual ligation hybridization assay extends the specificity of the hybridization ligation assay to a specific method for the parent compound. The DLA is intended to quantify the full-length, parent oligonucleotide compound only, with both intact 5′ and 3′ ends. A capture probe and a detection probe are ligated at the 5′ and 3′ ends of the analyte by the joint action of T4 DNA ligase and T4 polynucleotide kinase. The nuclease hybridization assay is a nuclease protection assay-based hybridization ELISA. In the nuclease hybridization assay, the oligonucleotide analyte is captured onto the solid support via a fully complementary cutting probe. After enzymatic processing by S1 nuclease, the free cutting probe and the cutting probe hybridized to metabolite, i.e., shortmers of the analyte are degraded, allowing signal to be generated only from the full-length cutting probe-analyte duplex.

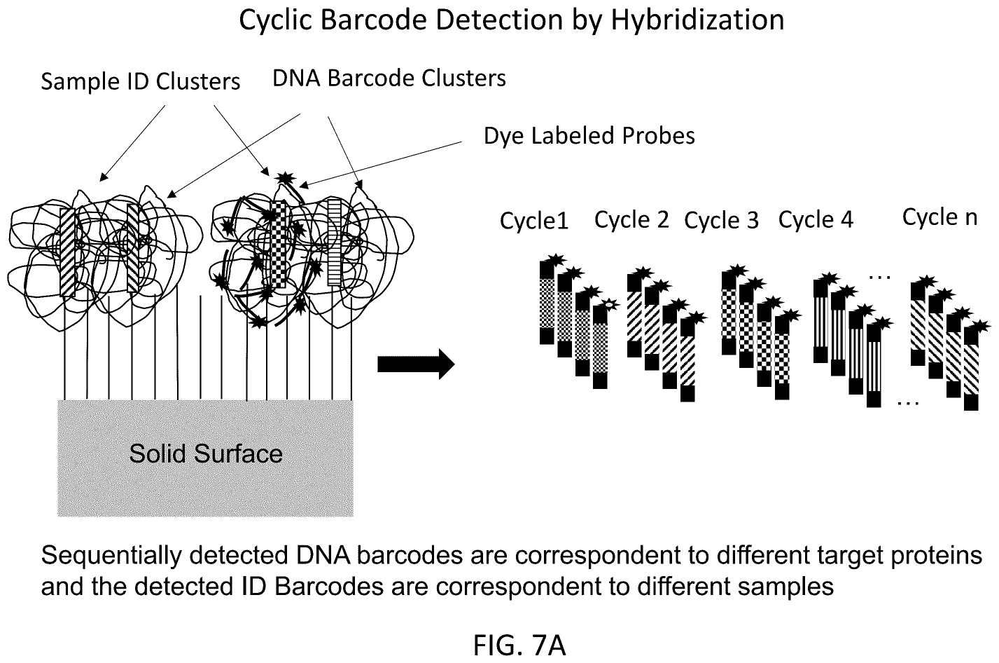

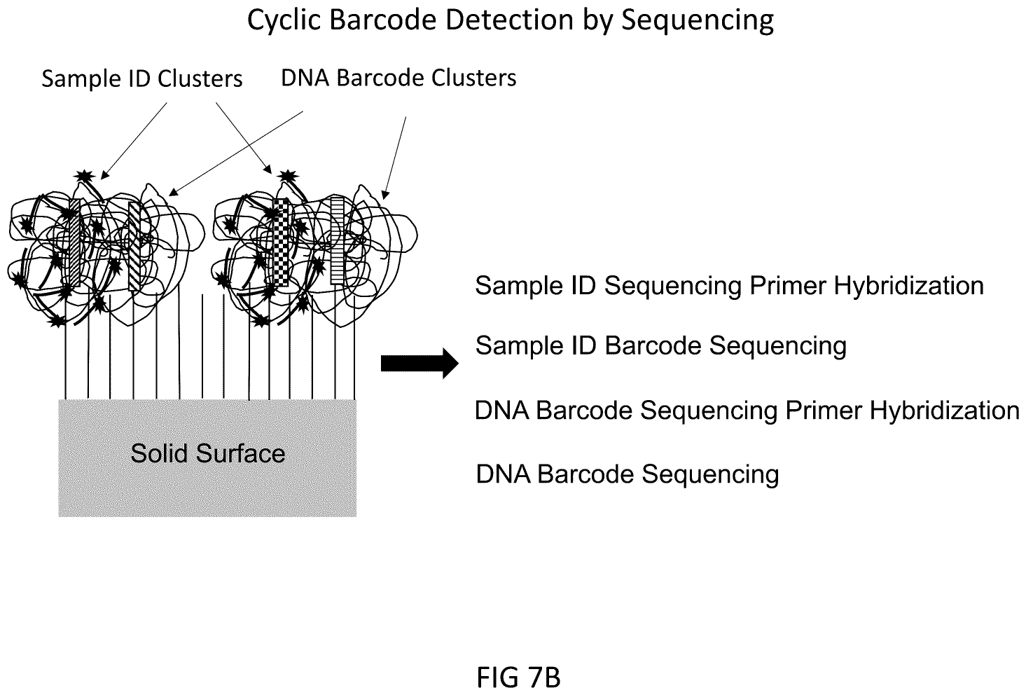

FIG. 7A illustrates cyclical barcode detection of the DNA strands on the solid surface using hybridization assay. An ID barcode probe is to decode sample information in the cluster and a barcode probe is to decode the protein barcode information of the extended coding DNA strand colocalized on the solid surfaces. A sample ID barcode probe is specific for a sample ID DNA strand and a protein barcode probe is a specific sequence of nucleic acids for a specific target protein. Using these ID barcode probes and the protein barcode probes, the information of samples and the information of target proteins can be sequentially decoded using hybridization assays.

In some embodiments, DNA barcode is cyclically decoded by sequencing. Both the sample ID DNA strands and the extended coding DNA strands on solid surface have sequencing priming sites. In some embodiments, the sample ID DNA strands and the extended coding DNA strands share the same sequencing primer. Using this single sequencing primer, the sample information of the sample ID DNA strands and the target protein information of the extended coding DNA strands can be sequentially decoded by sequencing in a stepwise manner. The information of the sample ID DNA strand includes, but not limited to, one or more unique molecular identifier (UMI) sequences, one or more spacer sequences, one or more sample ID sequence, one or more compartment sequences and or any combination thereof. The information of the extended coding DNA strands includes, but not limited to, one or more primer sequences, one or more unique molecular identifier (UMI), a coding sequences associated a specific target protein, one or more spacer sequences, one or more sample ID sequence, one or more compartment sequences, one or more sequencing cycle number sequences or any combination thereof. FIG. 7B illustrates that the sample ID DNA coding strands and the extended coding DNA strands have different sequencing primers. For example, the sample ID DNA strands have a specific sequencing primer and the extended coding DNA strands have another specific sequencing primer. Thus, after hybridizing the specific primer with the sample ID DNA strands, the sample information of the sample ID DNA strands can only be sequentially decoded by sequencing in a stepwise method. After hybridizing the specific primer with the extended coding DNA strands, the target protein information of the extended DNA strands can only be sequentially decoded by sequencing in a stepwise method.

In some embodiments, DNA barcodes are cyclically decoded after coding DNA strands are amplified into clusters in situ on solid surfaces. In some embodiments, these DNA barcodes are cyclically decoded at single molecule level without amplifying coding DNA strands into clusters on solid surfaces.

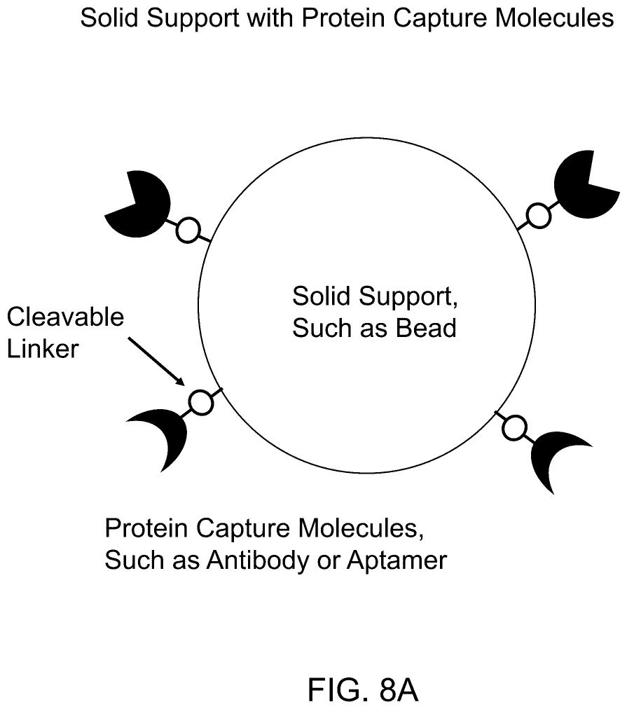

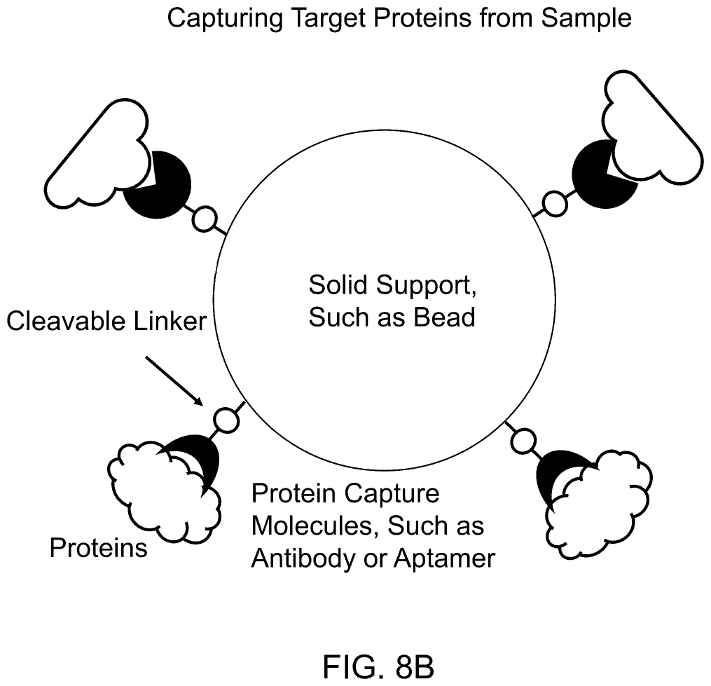

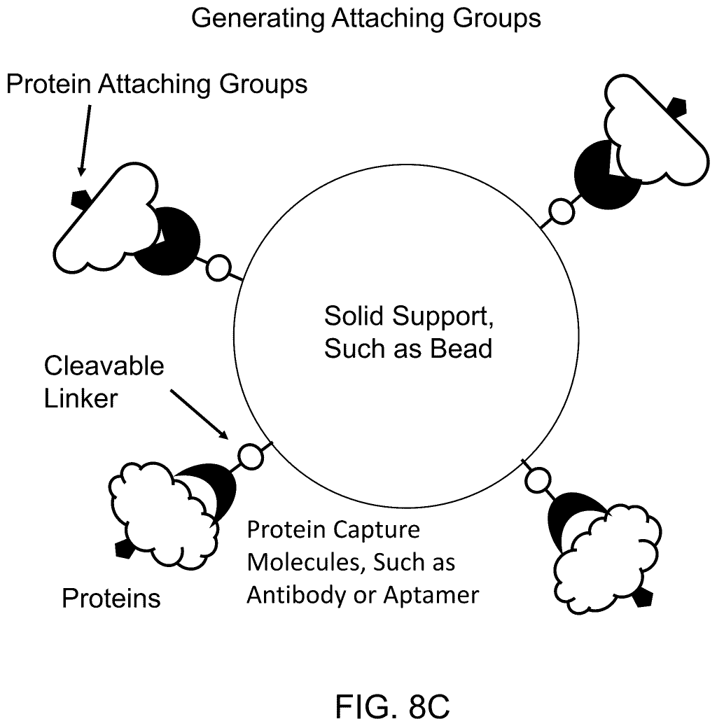

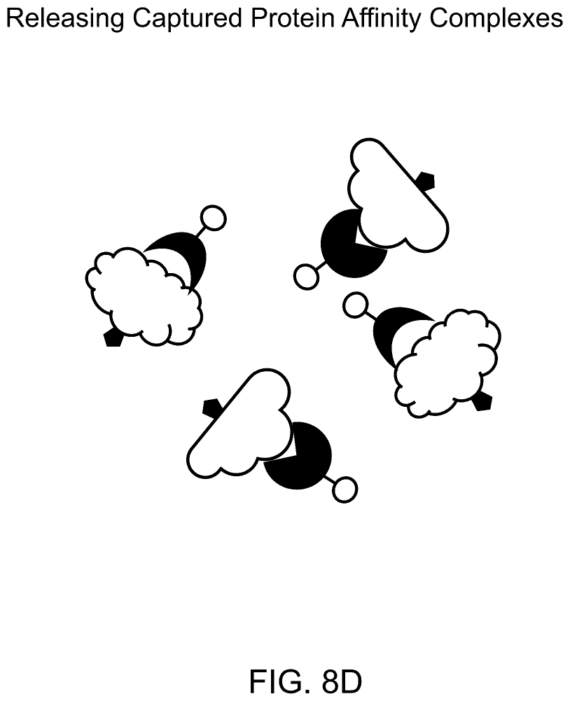

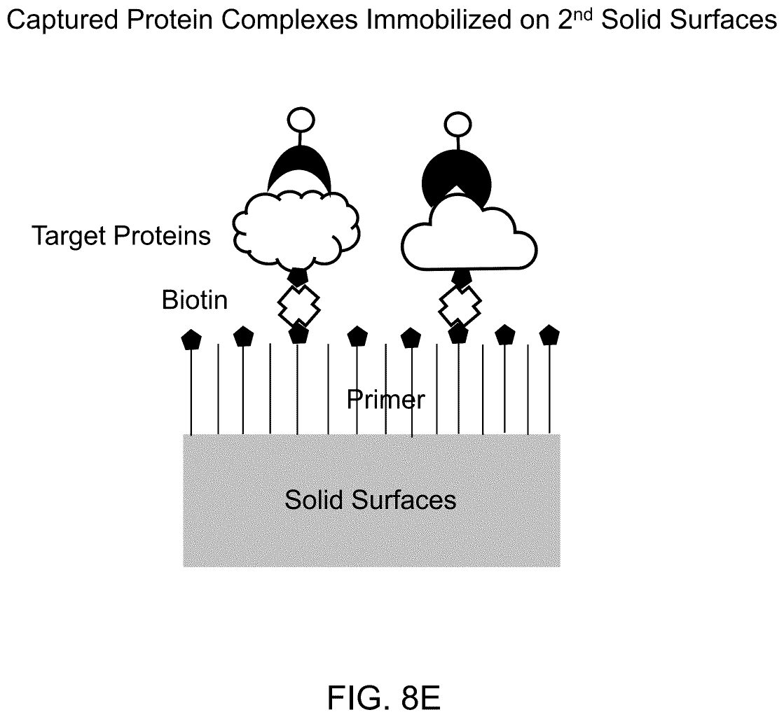

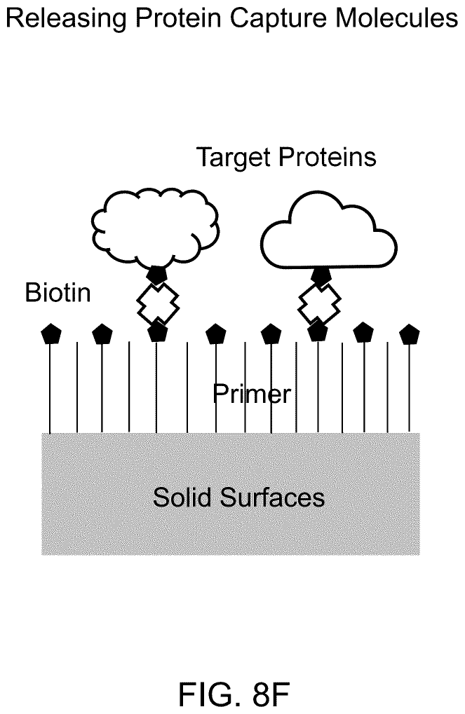

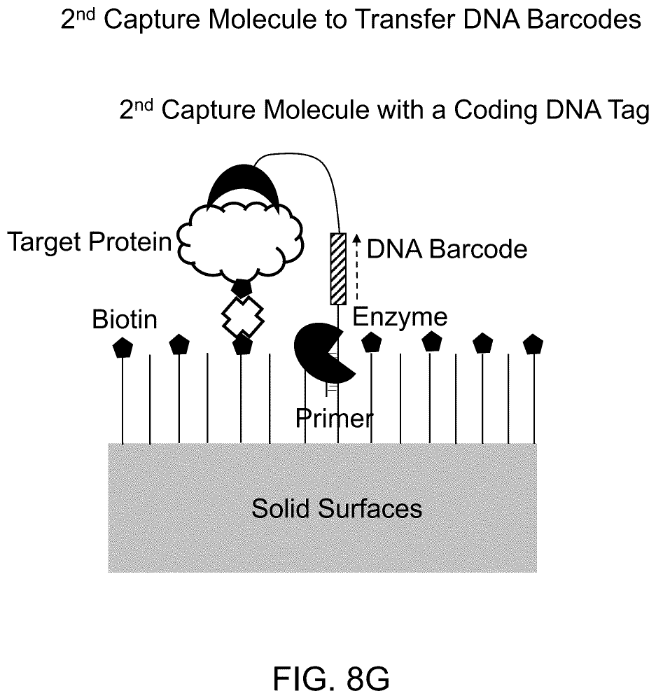

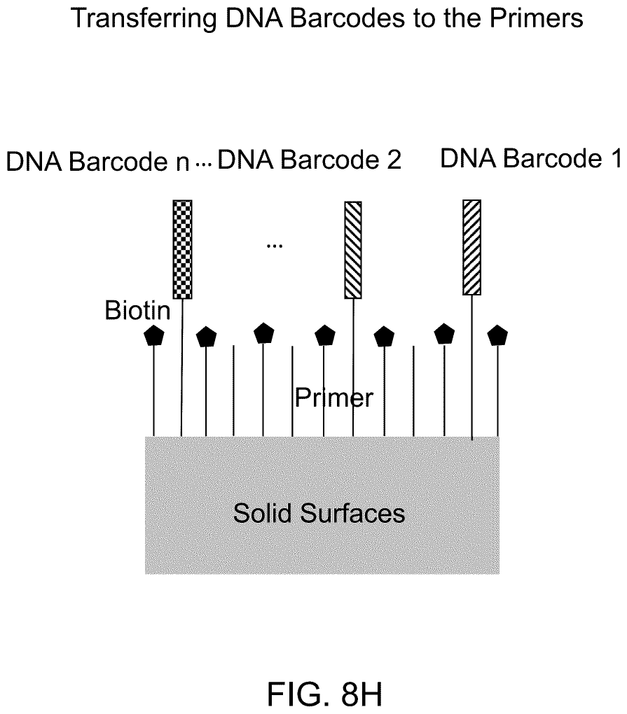

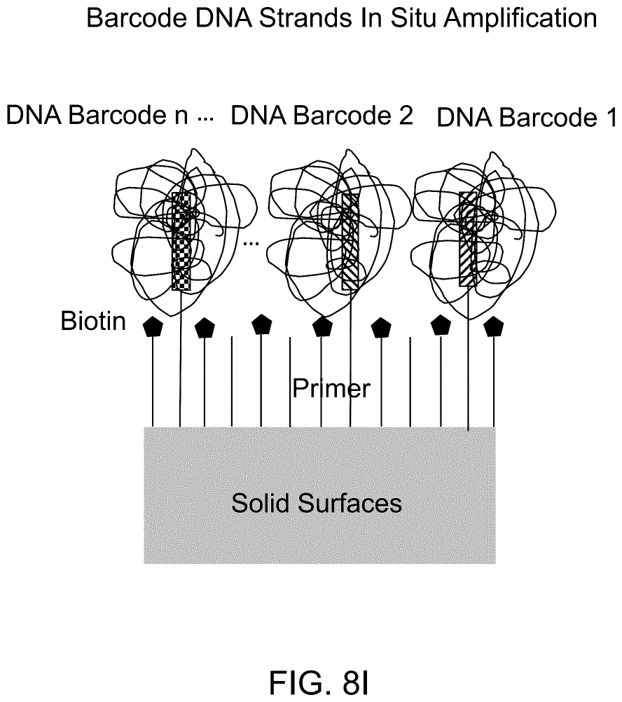

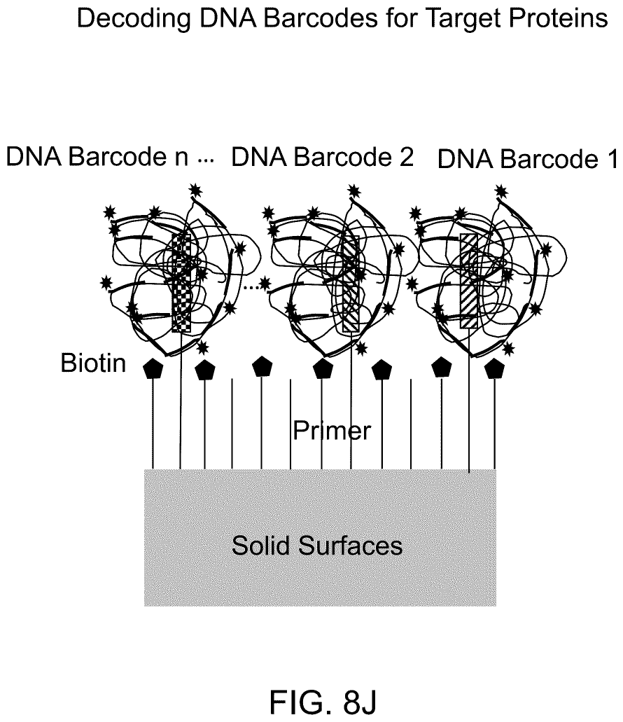

FIG. 8A to FIG. 8J illustrate an assay or a method for highly parallelized protein detection and analysis for single sample in one embodiment, comprising the steps of: (a) providing a 1^{st} capture molecule on 1^{st} solid surfaces through a cleavable linker; (b) capturing a target protein with the 1^{st} capture molecule on the 1^{st} solid surfaces; (c) generating an attaching group on the captured protein complex without disrupting the captured protein complex; (d) cleaving the captured protein complex and then immobilized on the 2^{nd} solid surfaces through the attaching group; (e) releasing the 1^{st} capture molecule; (f) contacting the immobilized protein with a 2^{nd} capture molecule with a coding DNA tag; (g) transferring the DNA barcode information and extending a primer on the 2^{nd} solid surface; (h) optionally amplifying the extended coding DNA strand into a cluster in situ on the 2^{nd} solid surfaces; (i) decoding the barcode DNA strands on the 2^{nd} solid surfaces.

The attaching group on the 1^{st} capture protein in step (c) includes, but is not limited to, primary amine, carboxylic acid, alkyne, acryloyl, allyl and aldehyde. The functional group on the 2^{st} solid surface in step (d) can covalently immobilize the protein complex on the surfaces through a free attaching group on the captured target protein. The surface functional group includes, but not limited to, aldehyde, oxime, hydrazone, hydrazide, alkyne, amine, azide, acylazide, acylhalide, nitrile, nitrone, sulfhydryl, disulfide, sulfonyl halide, isothiocyanate, imidoester, N-hydroxysuccinimide ester, pentynoic acid ester. In some embodiments, the protein complex is immobilized on the 2^{nd} solid surface through an affinity pair. The affinity pair includes, but not limited to, an antigen and an antibody against the antigen (including its fragments, derivatives, or mimetics), a ligand and its receptor, complementary strands of nucleic acids (e.g., Poly A or Ploy T), biotin and avidin (or streptavidin or neutravidin), lectin and carbohydrates, and vice versa.

A kinetic challenge is optionally inserted between step (c) and step (d) to remove or reduce the non-specific protein affinity complexes to improve the specificity of the assay. The kinetic challenge is performed by adding a competitor to the mixture containing the captured protein complexes and subsequently incubating the capture protein complexes in the competitor solution for a time less than or equal to the dissociation half-life of the protein affinity complexes, which can significantly increase the specificity of the detection assay.

### EXAMPLES

The following examples are provided for illustrative purposes only and are not intended to limit the scope of the invention as defined in the appended claims. The foregoing describes the disclosure with reference to various embodiments and examples. No particular embodiment, example, or element of a particular embodiment or example is to be constructed as a critical, required, or essential element or feature of any of the claims.

It will be appreciated that various modification and substitutions can be made to the disclosed embodiments without departing from the scope of the disclosure as set forth in the claim below. The specification is to be regarded in an illustrative manner, rather than a restrictive one, and all such modifications and substitutions are intended to be included within the scope of the disclosure. Accordingly, the scope of the disclosure may be determined by the appended claims and their legal equivalents, rather than by the examples. For examples, steps recited in any of the method or process claims may be executed in any feasible order and are not limited to an order presented in any of the embodiments, the examples or the claims.

### Example 1

### Capturing Targeted Proteins and DNA Barcode Transferring

A mixture of 1^{st} capture molecules, each with a 5′ biotin, a photocleavable group and a spacer (in that order from 5′ end) is immobilized on the streptavidin-coated beads and incubated with a protein mixture in a biological sample and the protein affinity complexes are formed. Following extensive washing of immobilized target protein, a biotinylated sample ID DNA tag is covalently attached with the immobilized protein through a chemically cleavable linker without disrupting the captured protein complex. The protein complexes are then released from the beads by photocleavage and diluted in a buffer containing dextran sulfate to selectively displace nonspecific interaction because of their faster dissociation rates.

After the kinetic challenge step, the protein complexes are captured on a streptavidin and primer mixed slide surfaces through the biotin molecule on the sample ID DNA tag of the target proteins and then washed again. The 1^{st} capture molecules are released by extensive high salt washing. A mixture of 2^{nd} capture molecules is incubated with these immobilized proteins and then protein complexes are formed again. The 2^{nd} capture molecule connects a coding DNA tag through a chemically cleavable linker. The coding DNA tag is ligated with an adjacent primer on the slide surfaces. Because both the coding DNA tag and the sample ID tag connect the immobilized protein complex through chemically cleavable linkers, the protein complex is released from the slide surfaces by chemical cleavage and then both the sample ID strand and the extended barcode DNA strand are left on the slide surfaces. After the extensive buffer washing, these DNA barcode strands, including the sample ID strand and the extended barcode DNA strand are ready for amplification on the slide surfaces.

### Example 2

### Cluster In Situ Amplification and DNA Barcode Sequencing

A mixture of circular templates is incubated with the sample ID strands and the extended barcode DNA strands on the surfaces for more than 20 mins. The circulate templates are hybridized with the sample ID strands and the extended barcode DNA strands on the slide surfaces. After extensive buffer washing, the amplification mixture with Phi29 enzyme flows in to initiate amplification reaction and then incubates for 20-60 mins. After the incubation, reaction stop buffer solution replaces amplification mixture to stop the amplification reaction. Following sequencing buffer washing, the slide surfaces have a plural of the barcode DNA clusters ready for sequencing.

A mixture of the sample ID sequencing primers is incubated and then annealed with the sample ID strands on the slide surfaces. Following a mixture of dye labeled dNTPs, the reaction is incubated for couple minutes and then wash away the reaction buffer and free dye labeled dNTPs for imaging. The pipeline analysis can analyze the images to decode the sample ID information. A mixture of the barcode sequencing primers is incubated and then annealed with the extended barcode DNA strands on the slide surfaces. Following a mixture of dye labeled dNTPs, the reaction is incubated for couple minutes and then wash away the reaction buffer and free dye labeled dNTPs for imaging. The pipeline analysis can analyze the images to decode the target protein information.

The sample ID barcode strand is colocalized with the extended barcode DNA strand derived from the same target protein on the slide surfaces. The distance of the sample ID barcode strand cluster and the extended barcode DNA strand cluster derived from the same target protein is closed and hard to optically resolved, so the detection signal from the sample ID barcode strand cluster is from the same spot in the image with the detection signal from the extended barcode DNA strand cluster derived from the same target protein. Based on this colocalization relation, each detected sample ID is correspondently assigned to the detected target proteins.

### REFERENCES

### U.S. Patent Documents

U.S. Pat. No. 6,511,809 B2 6/2003 Baez et al
U.S. Pat. No. 7,883,849 B2 8/2011 Dahl et al
U.S. Pat. No. 8,268,554 B2 9/2012 Schallmeriner et al
U.S. Pat. No. 8,404,830 B2 3/2013 Zichi et al
U.S. Pat. No. 9,404,919 B2 8/2016 Schneider et al
U.S. Pat. No. 9,435,810 B2 3/2014 Havranek et al
U.S. Pat. No. 9,551,032 B2 1/2017 Landegren et al
U.S. Pat. No. 9,732,341 B2 8/2017 Uhlmann et al
U.S. Pat. No. 10,233,488 B2 3/2019 Li et al
U.S. Pat. No. 10,371,634 B2 8/2019 Rothberg et al
U.S. Pat. No. 10,465,235 B2 11/2019 Gullberg et al
U.S. Pat. No. 10,612,093 B2 4/2020 Landegren et al
U.S. Pat. No. 10,655,163 B2 5/2020 Weissleder et al
U.S. Pat. No. 11,034,995 B2 6/2021 Soderberg et al
U.S. Pat. No. 11,435,358 B2 9/2022 Marcotte et al
U.S. Pat. No. 11,513,126 B2 11/2022 Chee et al
2007/0218503 A1 9/2007 Mitra et al
2014/0349860 A1 11/2014 Marcotte et al
2015/0087526 A1 1/2013 Hesselberth et al
2020/0286584 A1 8/2019 Patel et al
2020/0209256 A1 12/2019 Reed et al
2020/0231956 A1 3/2020 Callewaert et al
2020/0217853 A1 7/2020 Estandian et al
2020/0219590 A1 7/2020 Reed et al
2020/0348307 A1 11/2020 Beierle et al
2020/0395099 A1 12/2020 Meyer et al
2021/0079557 A1 3/2021 Pawlosky et al
2021/0239705 A1 8/2021 Mallick et al
2021/0285941 A1 9/2021 Luo et al
2021/0405058 A1 12/2021 Chee et al
2021/0396762 A1 12/2021 Chee et al
2022/0290218 A1 3/2022 Aksel et al
2022/0127754 A1 4/2022 Verespy III et al
2022/0214353 A1 7/2022 Chee et al
2022/0290218 A1 9/2022 Aksel et al
2023/0104998 A1 4/2023 Estandian et al

### Foreign Patent Documents

| | | |
|---|---|---|
| WO | WO 2010/065531A1 | December 2008 |
| WO | WO 2010/065522A1 | December 2008 |
| WO | WO 2013/112745A1 | January 2012 |
| WO | WO 2021/236983A2 | May 2020 |
| WO | WO 2022/040098A1 | August 2020 |
| WO | WO 2023/049177A1 | March 2023 |
| WO | WO 2023/196642A1 | October 2023 |

### Other Publications

Gold et al., “Aptamer-Based Multiplexed Proteomic Technology for Biomarker Discovery”, PLOS One, 5(12), e15004, (2010).
Rohloff et al., “Nucleic Acid Ligand with Protein-Like Side Chains: Modified Aptamers and Their Use as Diagnostic and Therapeutic Agents”, Molecular Therapy-Nucleic Acids, 3, (2014).
Wik et al., “Proximity Extension Assay in Combination with Next Generation Sequencing for High-throughput Proteome-wide Analysis”, Mol Cell Proteomics, 20, 100168, 1-16, (2021).
Feng et al., “NULISA: A Proteomic Liquid Biopsy Platform with Attomolar Sensitivity and High Multiplexing”, Nature Communications, 14, 7238, 1-14, (2023).
Groote et al., “Highly Multiplexed Proteomic Analysis of QuantiFERON Supernatants to Identify Biomarkers of Latent Tuberculosis Infection”, J. of Clinical Microbiology, Vol. 55, 391-402, (2017).
Green et al., “Aptamers as Reagent for High-Throughput Screening”, BioTechniques, 30, 1094-1110, (2001).
Agasti et al., “Photocleavable DNA Barcode-antibody Conjugates Allow Sensitive and Multiplexed Protein Analysis in Single Cell”, J. Am. Chem. Soc., 134 (45), 18499-18502, (2012).
Ma et al., “Isothermal Amplification Method for Next Generation Sequencing”, Proc. Natl. Acad. Sci., 110(35), 14320-14323, (2013).
Drmanac et al., “Human Genome Sequencing Using Unchained Base Read on Self-assembling DNA Nanoarrays”, Science, 327(5961), 78-81, (2010).
Rothberg et al., “An Integrated Semiconductor Device Enabling Non-Optical Genome Sequencing”, Nature, 475, 348-352, (2011).
Arslan et al., “Sequencing by Avidity Enable High Accuracy with Low Reagent Consumption”, Nature Biotech., (2023).
Bentley et al., “Accurate whole human genome sequencing using reversible terminator chemistry”. Nature, 456, 53-59, (2008).
Zhao et al., “Single Molecule Sequencing of the M13 Virus Genome Without Amplification.”, PLOS one, 1-9, (2017).
Levene et al., “Zero-Mode Waveguides for Single-Molecule Analysis at High Concentrations.” Science, 299, 682-686, (2003).
Jepsen et al., “Locked Nucleic acid: A Potent Nucleic Acid Analog in Therapeutic and Biotechnology.”, Oligonucleotides, 14, 130-146, (2004).
Siddiquee et al., “A Review of Peptide Nucleic Acid.”, Adv. Tech. Biol. Med, 2, 1000131, (2015).
Gunderson et al., “Mutation Detection by Ligation to Complete N-mer DNA Arrays.”, Genome Res., 8, 1142-1153, (1998).
El-Sagheer et al., “Rapid Chemical Ligation of Oligonucleotides by the Diels-Alder Reaction.”, 9, 232-235, (2011).

What is claimed is:

1. A method for protein detection and analysis, the method comprising the steps of:
(a) binding a target protein with a capture molecule with a DNA coding tag;
(b) transferring the barcode information of the DNA coding tag to a primer and then forming an extended DNA tag on the solid surfaces;
(c) decoding DNA strands on the solid surfaces.

2. The method of claim 1, wherein in step (a) the capture molecule is a protein based binding molecule or a nucleic acid based aptamer.

3. The method of claim 1, wherein in step (a) the DNA coding tag comprises one or more primer sequences, one or more unique molecular identifier (UMI), a coding sequences associated specific target protein, one or more spacer sequences, one or more sample ID sequence, one or more compartment sequences, or any combination thereof.

4. The method of claim 1, wherein in step (a) the DNA coding tag connects an attaching element or a cleavable element or combination of these two elements.

5. The method of claim 1, wherein in step (b) the transferring method is ligation or extension.

6. The method of claim 1, wherein in the step (b) the extended DNA strand is colocalized with the associated protein on the solid surfaces.

7. The method of claim 6, wherein the distance of the extended DNA strand and the colocalized target protein is, 1 nm, 2 nm, 3 nm., 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

8. The method of claim 1, wherein in step (b) the primer comprises a priming site for amplification, an adaptor sequence that anneal to complementary oligonucleotides on a DNA tag of a binding molecule, a sequencing priming site, or a combination thereof.

9. The method of claim 8, wherein the primer might have low melting temperature property.

10. The method of claim 9, wherein the low melting temperature of the universal primer is 25° C., 30° C., 35° C., 40° C., 45° C., 50° C., 55° C., 60° C. and above, or any temperature between two aforementioned temperatures.

11. The method of claim 1, wherein in step (c) the decoding method of the immobilized DNA clusters on the solid surfaces is hybridization assay or sequencing.

12. The method of claim 11, wherein the sequencing method is next generation sequencing or single molecule sequencing.

13. The method of claim 12, wherein the sequencing method is optical fluorescence based next generation sequencing or single molecule sequencing.

14. The method of claim 1 further comprising of the step amplifying the extended DNA strands into clusters in situ on the solid surfaces between step (b) and step (c).

15. The method of claim 14, wherein the in-situ amplification reaction of the extended DNA strands is isothermal amplification or PCR.

16. The method of claim 15, wherein the isothermal amplification method comprises template walking, RPA, RCA, LAMP, SDA or MDA.

17. The method of claim 15, wherein PCR comprises bridge PCR.

18. The method of claim 14, wherein the amplified clusters derived from the same target protein are colocalized on the solid surfaces.

19. The method of claim 18, wherein the distance of the colocalized DNA clusters derived from the same protein, is 1 nm, 2 nm, 3 nm., 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

20. A method for protein detection and analysis, the method comprising the steps of:
(a) providing a 1^{st} capture molecule that is immobilized on 1^{st} solid surfaces through a cleavable linker;
(b) binding a target protein with the 1^{st} capture molecule on the 1^{st} solid surfaces;
(c) generating an attaching group on the captured target protein;
(d) cleaving the linker and then releasing the complex molecule of the target protein;
(e) immobilizing the complex molecule on the 2^{nd} solid surfaces through the attaching group on the target protein;
(f) releasing the 1^{st} capture molecule from the immobilized complex molecule on the 2^{nd} solid surfaces;
(g) contacting the target protein with a 2^{nd} capture molecule with a DNA coding tag;
(h) transferring barcode information from the DNA coding tag of the 2^{nd} capture molecule to a primer and then forming an extended DNA strand on 2^{nd} the solid surfaces;
(i) decoding the DNA strands on the 2^{nd} solid surfaces.

21. The method of claim 20, wherein in step (a) the solid support comprises a bead, a porous bead, a glass surface, a silicon surface, a metal surface, or a plastic surface.

22. The method of claim 20, wherein in step (a) the 1st capture molecule is a protein based binding molecule or a nucleic acid based aptamer.

23. The method of claim 20, wherein in step (a) the cleavable linker is a PC linker, a chemically cleavable linker, or an enzymatically cleavable linker.

24. The method of claim 20, wherein in step (c) the attaching group is primary amine, carboxylic acid, alkyne, acryloyl, allyl or aldehyde.

25. The method of claim 20 further comprising of the step attaching a sample ID DNA tag with the target protein through an attaching group between step (c) and step (d).

26. The method of claim 25, wherein the sample ID DNA tag comprises one or more unique molecular identifier (UMI) sequences, one or more spacer sequences, one or more sample ID sequence, one or more compartment sequences and/or any combination thereof.

27. The method of claim 25, wherein the sample ID DNA tag connects an attaching element or a cleavable element or combination of these two elements.

28. The method of claim 20 further comprising of the step of a kinetic challenge between step (c) and step (d).

29. The method of claim 20 further comprising of the step pooling the released protein complexes from different samples together between step (d) and step (e).

30. The method of claim 20, wherein in step (e) the complex molecule is immobilized on the 2^{nd} solid surfaces through an attaching group or a sample ID DNA tag.

31. The method of claim 20, wherein in step (f) the 1^{st} capture molecules are released by one or more of following treatment: high salt, high pH, low pH or evaluated temperature.

32. The method of claim 20, wherein in step (g) the coding DNA tag comprises one or more primer sequences, one or more unique molecular identifier (UMI), a coding sequences associated specific proteins, one or more spacer sequences, one or more sample ID sequence, one or more compartment sequences, one or more sequencing cycle number sequences or any combination thereof.

33. The method of claim 20, wherein in step (g) the coding DNA tag connects with an attaching element or a cleavable element or combination of these two elements.

34. The method of claim 20, wherein in step (h) the transferring method is ligation or extension.

35. The method of claim 20, wherein in the step (h) the extended DNA strand and the sample ID DNA coding strand derived from the same target protein are colocalized on the 2^{nd} solid surfaces.

36. The method of claim 35, wherein the distance of these colocalized DNA strands is 1 nm, 2 nm, 3 nm., 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.

37. The method of claim 20, wherein in step (h) the primer comprises a priming site for amplification, an adaptor sequence that anneal to complementary oligonucleotides on a DNA tag of a binding molecule, a sequencing priming site, or a combination thereof.

38. The method of claim 37, wherein the primer has low melting temperature property.

39. The method of claim 38, wherein the low melting temperature of the primer is 25° C., 30° C., 35° C., 40° C., 45° C., 50° C., 55° C., 60° C. and above, or any temperature between two aforementioned temperatures.

40. The method of claim 20 further comprising of a step amplifying immobilized DNA strands into clusters in situ on the 2^{nd} solid surfaces between step (h) and step (i).

41. The method of claim 40, wherein the in-situ amplification reaction of the DNA strands is isothermal amplification or PCR.

42. The method of claim 41, wherein the isothermal amplification method comprises template walking, RPA, RCA, LAMP, SDA or MDA.

43. The method of claim 41, wherein PCR comprises bridge PCR.

44. The method of claim 40, wherein these amplified clusters derived from the same target protein are colocalized on the 2^{nd} solid surfaces.

45. The method of claim 44, wherein the distance of the colocalized DNA clusters derived from the same target protein, is 1 nm, 2 nm, 3 nm, 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, or any distance between two aforementioned distances.
